SEQUENCE LISTING (6 sequences)

SEQ ID NO: 1 - DNA, 24 bp, Artificial
  Primer Oct4 F1
gttctctttggaaaggtgttcagc

SEQ ID NO: 2 - DNA, 24 bp, Artificial
  Primer Oct4 R1
actcgaaccacatccttctctagc

SEQ ID NO: 3 - DNA, 25 bp, Artificial
  Primer Oct4 S1
cttgctgcagaagtgggtggaggaa

SEQ ID NO: 4 - DNA, 21 bp, Artificial
  Primer Oct4 A1
ctgcagtgtgggtttcgggca

SEQ ID NO: 5 - DNA, 18 bp, Artificial
  Primer Dog-POU5F1-S1
tgacgacaacaaaaatct

SEQ ID NO: 6 - DNA, 18 bp, Artificial
  Dog-POU5F1-A1
caggcatgtgttctccag

(19) United States
(12) Patent Application Publication
(10) Pub. No.: US 2007/0053890 A1
(43) Pub. Date: Mar. 8, 2007

(54) Canine embryonic stem cells

(75) Inventors: Suzana Rosic-Kablar, Toronto (CA); Margaret R. Hough, Toronto (CA); Andras Nagy, Toronto (CA)

Correspondence Address: MERCHANT & GOULD PC, P.O. BOX 2903, MINNEAPOLIS, MN, 55402-0903, US

(73) Assignees: Sunnybrook and Women's College Health Sciences Centre, Toronto (CA); Mount Sinai Hospital, Toronto (CA)

(21) Appl. No.: 10/551,153

(22) PCT Filed: Mar. 26, 2004
(86) PCT No.: PCT/CA04/00456

§ 371 Date: Oct. 9, 2006

Related U.S. Application Data
(60) Provisional application No. 60/458,863, filed on Mar. 28, 2003.
(60) Provisional application No. 60/526,385, filed on Dec. 1, 2003.

(51) Int. Cl. A61K 48/00; C12Q 1/68; G06Q 50/00; C12N 5/06; C07H 21/04

(52) U.S. Cl. 424/93.21; 514/44; 435/6; 435/350; 705/2; 536/23.1

(57) ABSTRACT

The invention relates to canine embryonic stem cells, methods of cultivation and propagation of the cells, and production of differentiated cells. The embryonic stem cells may be obtained by isolating a canine embryo, culturing the embryo in the presence of a feeder layer and one or more proliferation agent, removing a blastocyst outgrowth, and culturing the outgrowth in the presence of a fresh feeder layer.

### FIELD OF THE INVENTION

The invention relates to the field of in vitro culture of stem cells and methods of producing the cells. More particularly the invention relates to canine embryonic stem cells and cell lines.

### BACKGROUND OF THE INVENTION

The establishment of embryonic stem (ES) cell lines has brought great promise and opportunities for regenerative medicine and pharmaceutical research. Embryonic stem cells are derived from embryonic sources and are pluripotent i.e. they possess the capability of developing into a wide variety of different cell types, and tissues and organs.

Procedures for the development of embryonic stem cell lines for different species poses challenges due to differences in the pattern of embryonic development in different species. Different strategies are required in order to prepare embryonic stem cells from a particular species. More specifically, careful timing is required in the isolation of embryonic stem cells from a species so that the inner cell mass (ICM) cells remain pluripotent and are not influenced by differentiation elements. Culture strategies also have to be defined to sufficiently allow expansion of ES cells and prevent ES cell differentiation in order to establish the cell lines.

Pluripotent embryonic stem cell lines have been derived from preimplantation embryos of mice (Evans et al, Nature 292:154-139 1981; Martin, Proc. Natl. Acad. Sci. USA 78:7634-7638, 1981) and several domestic and laboratory animal species (Evans et al, Theriogenology 33(1): 125-128, 1990; Notarianni et al, J. Reprod. Fertil. 41 (Suppl.) 51:-56, 1990; Giles et al., Mol. Reprod. Dev. 33:418-431, 1992; Sukoyan et al., Mol. Reprod. Deve. 36: 424-433; 1993; Sukoyan et al., Mol. Reprod. Dev. 33:418-431, 1992; Sukoyan, et al., Mol Reprod. Dev. 36:148-158, 1993, Iannaccone et al Dev. Biol. 163: 288-292, 1994; U.S. Patent Application 20020187549). Pluripotent embryonic stem cell lines have also been described for primates and humans (U.S. Pat. No. 6,331,406; US 20030008392; US 20020160509).

To date, there have been no reports for the establishment of canine embryonic cells or cell lines. Methods which would allow production of canine embryonic cells and cell lines would permit easier study of canine development, provide a preclinical model for the development of human therapies, permit the development of conditions for in vitro differentiation of ES cells to cell derivatives of all three embryonic germ layers, and the use of canine cell lines would enable the development of cell cultures for transplantation, development of procedures for cloning purebred dogs, and the development of transgenic animals, in particular animal models of disease.

The citation of any reference herein is not an admission that such reference is available as prior art to the instant invention.

### SUMMARY OF THE INVENTION

Applicants were able to define conditions for isolation of embryos and stem cells from canines and for establishing canine embryonic stem cells and cell lines. More particularly, Applicants identified appropriate culture conditions and determined embryonic developmental stages that enable maintenance and expansion of canine embryonic stem cells.

The present invention provides cells exhibiting a canine embryonic stem cell phenotype, and cell lines comprising cells exhibiting a canine embryonic stem cell phenotype.

The invention further relates to a purified preparation comprised or enriched for canine embryonic stem cells that are capable of indefinite proliferation in vitro in an undifferentiated state. A preparation of canine embryonic stem cells may also be characterized by being immunoreactive with markers for embryonic stem cells, preferably canine embryonic stem cells.

Canine embryonic stem cells of the invention may be induced to differentiate into cells of a variety of lineages in vitro or in vivo. In an embodiment, the invention relates to a purified canine embryonic stem cell preparation of the invention (preferably cultured in vitro) induced to differentiate into cells of various lineages. A differentiated cell preparation is characterized by expression of genetic markers of various cell lineages

In an embodiment, the invention provides cells differentiated in vitro from a canine embryonic stem cell of the invention. In addition, a committed progenitor cell capable of giving rise to a mature somatic cell is provided.

Embryonic stem cells or cells differentiated or derived therefrom according to the invention can be cultured either transiently or maintained as a cell line. Thus, the present invention also relates to a cell line comprising canine embryonic stem cells, or cells differentiated or derived therefrom.

Cells, cell lines, and cell preparations of the invention may be derived from or comprised of cells that have been genetically modified either in nature or by genetic engineering techniques in vivo or in vitro.

In an aspect of the invention a method is provided for producing canine embryonic stem cell lines that exhibit a canine embryonic cell phenotype.

The invention relates to a method for obtaining a purified canine embryonic cell line, comprising the steps of culturing inner cell mass (ICM) cells from a canine embryo under conditions to promote proliferation of undifferentiated cells. The method may additionally comprise inducing differentiation of the stem cells.

In an aspect of the invention a method is provided for obtaining cells exhibiting a canine embryonic stem cell phenotype. Cells exhibiting a canine embryonic stem cell phenotype may be isolated by (a) obtaining a canine embryo; (b) culturing inner cell mass (ICM) cells from the canine embryo under conditions which promote proliferation of undifferentiated stem cells; and (c) recovering stem cells.

In an aspect of the invention, the method comprises (a) isolating a canine embryo, (b) culturing the embryo in the presence of a feeder layer and one or more proliferation agents, (c) removing a blastocyst outgrowth and transferring to fresh feeder layers, and (d) selecting embryonic stem like cell colonies and subculturing the colonies. The invention also contemplates cell preparations or lines derived at all stages of development under the same culture conditions.

In an embodiment of the invention, a method of producing cells exhibiting a canine embryonic stem cell phenotype is provided comprising: (a) obtaining a canine embryo at a morula to expanded blastocyst stage; (b) removing inner cell mass (ICM) cells from the canine embryo; (c) culturing ICM cells in the presence of a feeder layer and one or more proliferation agent to promote proliferation of undifferentiated stem cells; and (c) recovering stem cells. The method may additionally comprise removing an outgrowth comprising ES-like cell colonies, dissociating the outgrowth, transferring to fresh feeders for expansion of colony numbers and selecting embryonic stem cell like colonies and culturing the colonies.

Stem cells obtained using a method of the invention may be passaged for several months in culture.

The invention also contemplates embryonic stem cells isolated from in vitro treatment of canine blastocysts. The invention further contemplates canine embryonic stem cells produced by a method of the invention. The resulting stem cells preferably resemble canine embryonic cells in morphology, biochemical histotype and in pluripotencty.

The invention also provides canine transgenic cells, cell lines, or tissues using the canine embryonic stem cells of the invention.

Stem cells of the invention may be used in genetic transformation techniques and may be used in the creation of embryos and to produce a genetically transformed animal by embryo transfer. Thus, the invention further provides an embryo (preferably an early stage embryo, for example, a morula to expanded blastocyst) to which has been introduced one or more canine embryonic stem cells of the invention; an embryonic stem cell to which has been introduced by nuclear transfer a nucleus of an embryonic stem cell of the invention; and a chimeric animal which is the progeny of such a blastocyst or embryonic stem cell.

In an aspect the invention provides a method comprising introducing by nuclear transfer into an embryonic cell a nucleus of a stem cell of the invention.

In another aspect the invention provides a method comprising introducing to the uterus of a pseudo-pregnant foster mother animal a viable embryo obtained using a blastocyst comprising one or more stem cells according to the invention, or an embryonic cell comprising a nucleus of a stem cell according to the invention.

The invention still further provides cells that exhibit a canine embryonic cell phenotype or stem cells derived therefrom of restricted developmental lineage for transplantation.

The invention also provides pharmaceutical products produced by the cells, cell lines, or cell preparations of the present invention, or mitotic or differentiated cells that are progeny of the cells.

Cells, cell lines, and cell preparations of the invention may be used in both cell therapies and gene therapies aimed at alleviating disorders and diseases. The invention contemplates a method of treating a subject with a condition comprising transferring to a patient an effective amount of cells of the invention.

The cells, cell lines, and cell preparations of the invention may be used as immunogens (or tolerizing agents) that are administered to a heterologous recipient.

The cells, cell lines, and cell preparations of the invention may be used to prepare model systems of disease, in particular canine and human diseases. The cells, cell lines, and cell preparations of the invention can also be used to produce growth factors, hormones, etc.

The invention also contemplates a pharmaceutical composition comprising cells, cell lines, and cell preparations of the invention, and a pharmaceutically acceptable carrier, excipient, or diluent. A pharmaceutical composition may include a targeting agent to target cells to particular tissues or organs.

Cells, cell lines, and cell preparations of the invention may be used to screen for potential therapeutics that modulate development or activity of such cells or cells differentiated therefrom.

In an aspect, the invention provides a method for screening compounds including small molecules that affect the function of cells of the invention. The method includes incubating components comprising a test compound and at least one cell of the invention under conditions sufficient to allow the components to interact; and determining the effect of the compound on a function of a cell before and after incubating with the test compound. A function of a cell of the invention may be modulated (e.g. inhibited or stimulated) by the test compound. By way of example, cell differentiation, gene expression, production of growth factors, response to growth factors, and cell membrane permeability may be modulated.

The invention also relates to a method for conducting a regenerative medicine business. Still further the invention relates to a method for conducting a stem cell business involving identifying agents that affect the proliferation, differentiation, function, or survival of canine embryonic stem cells of the invention. An identified agent(s) can be formulated as a pharmaceutical preparation, and manufactured, marketed, and distributed for sale.

In another aspect, the invention contemplates methods for influencing the proliferation, differentiation, or survival of cells of the invention by contacting the cells with a test agent.

The invention also contemplates a method of treating a subject comprising administering an effective amount of an agent identified in accordance with a method of the invention to a patient with a disorder affecting or involving the proliferation, differentiation, function, or survival of cells of the invention.

The invention also contemplates a method for conducting a drug discovery business comprising identifying factors or agents that influence the proliferation, differentiation, function, or survival of cells of the invention, and licensing the rights for further development.

The invention further contemplates a method of providing drug development wherein cells of the invention or mitotic or differentiated progeny thereof are used as a source of biological components of cells in which one or more of these biological components are the targets of the drugs that are being developed.

The invention also relates to methods of providing a bioassay.

In an aspect, the invention features a kit including cells generated using a method of the invention, or a mitotic or differentiated cells that are progeny of the cells.

The invention is also directed to a kit for transplantation of cells comprising a flask with medium and cells of the invention.

The invention also relates to a method of using the cells, cell lines, and cell preparations in rational drug design.

In an aspect, the invention relates to a kit for rational drug design comprising cells obtained by a method of the invention. In an embodiment, the kit comprises cells and instructions for their use in toxicity assays.

Still another aspect of the invention is a kit for producing cells of the invention, or for producing an expanded stem cell preparation.

The invention also provides primers that hybridize to an Oct4 canine nucleotide sequence. In particular, the invention provides a primer comprising the sequence of SEQ ID NO. 1, 2, 5 or 6.

These and other aspects, features, and advantages of the present invention should be apparent to those skilled in the art from the following drawings and detailed description.

### DESCRIPTION OF THE DRAWINGS

The invention will now be described in relation to the drawings in which:

FIG. 1 are photographs of (A) an embryo-derived outgrowth 5-10 days after the zona pellucida was cut open with a fine blade;) Low (B) and high power (C) magnification of canine ES colonies at passage 6.

FIG. 2 illustrates the morphology of an established Canine ES cell line. A phase contrast image of undifferentiated ES-like colonies well distinguished from the MEFs (A and B) and a higher magnification view (C and D) of tightly packed colony and cells with prominent nucleoli. At least two phenotypically distinct canine ES colonies can be identified: (A) Small, spherical, 3-D-like ES colonies and (B) large colonies with a more flattened appearance with well defined peripheral edges. Transfer of single cell suspensions or small clumps of cells from ES colonies to a sparse layer of MEFs resulted in the formation of structures resembling embryoid bodies (EBs) (E). (F) Low and (G and H) high magnification of cystic formations developing one week after transfer to non-coated culture dishes.

FIG. 3 illustrates the optimization of canine Oct4 RT-PCR and nested PCR. (A-C) Oct4 PCR amplification of cDNA generated from total RNA isolated from two early passage canine ES cell lines, murine ES cells and murine trophoblast stem (TS) cells. (A) PCR amplification of canine Oct4 in early passage ES cells using primer pairs Oct4S1 and Oct4R1. (B) PCR amplification of murine Oct4 sequences in ES and TS cell lines using Oct4 specific primers Oct4S1 and Oct4R1. (C) Nested PCR using Oct4S1 and Oct4A1 primer pairs and the PCR generated Oct4 fragment generated in figure A. (D-E) DNA sequence analysis of the canine Oct4 fragment amplified by nested PCR.

FIG. 4 shows Oct 4 expression in canine embryonic stem cells. Lane 1-2: Canine ES cells: Line 1, passage 1; Lane 3-4: Canine ES cells, Line 1, passage 10: Lane 5-ES cells, Line 1 passage 10 (RNA only) Lane 6. Negative control (H₂0).

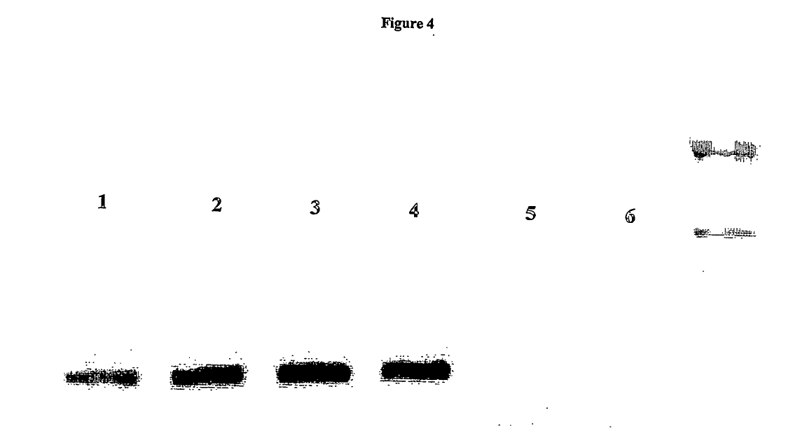

FIG. 5 shows (A) SSEA-4 and (B) TRA-1-60 expression in cells of canine ES colonies.

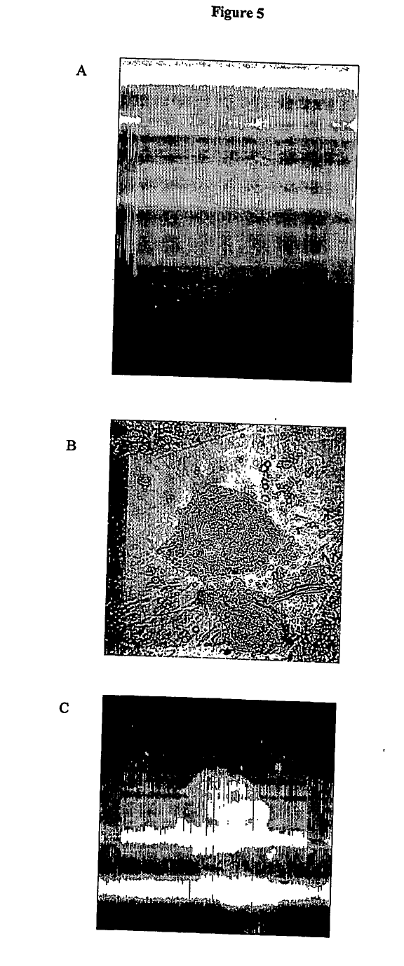

FIG. 6 Alkaline phosphatase expression in canine and murine ES cells. (A) Unstained mouse ES cells; (B) Mouse ES colony, and (C) Canine ES colony stained for expression of alkaline phosphatase.

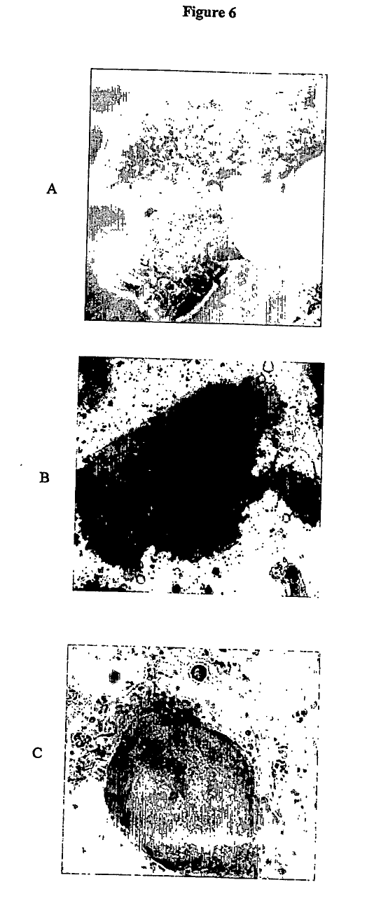

FIG. 7 depicts the hatching of cells of the inner cell mass of canine blastocysts on canine feeder cells. The canine feeder layer supported the hatching of expanded canine blastocysts but were unable to support canine ES cell proliferation in an undifferentiated state.

FIG. 8 shows the generation of canine ES cells on mouse feeder cells. Day 0: Morula; Day 1: Blastocyst showing cells of inner cell mass and blastocoel; Day 5: Expanded blastocyst; Day 12: Hatching; Day 19: Hatched ES cells before transfer to fresh MEFs; Day 27: ES colony growing on MEFs; Day 30: ES colonies growing on gelatinized plate with canine embryo-derived trophoblast-like cells; Day 30: High power magnification of ES colony.

FIG. 9 shows the in vitro differentiation of canine ES cells to endothelial and neuronal cells. (A) Differentiation of EBs to endothelial cells as indicated by morphological appearance and reactivity to the endothelial cell specific antigen; CD31. (B) In vitro differentiation of EBs to neuronal cells identified on the basis of morphological appearance.

### DETAILED DESCRIPTION OF A PREFERRED EMBODIMENT

In accordance with the present invention there may be employed conventional molecular biology, microbiology, and recombinant DNA techniques within the skill of the art. Such techniques are explained fully in the literature. See for example, Sambrook, Fritsch, & Maniatis (Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984). The invention may also employ standard methods in immunology known in the art such as described in Stites et al. (eds) Basic and Clinical Immunology, 8^{th} Ed., Appleton & Lange, Norwalk, Conn. (1994) and Mishell and Shigi (eds), Selected Methods in Cellular Immunology, W. H. Freeman and Co., New York (1980).

For convenience, certain terms employed in the specification and claims are collected here.

“Subject” or “patient” refers to an animal, preferably a mammal, to whom treatment, including prophylactic treatment, with the cells, cell preparations, and compositions of the present invention, is provided. For treatment of those conditions or disease states that are specific for a specific animal, the terms refer to that specific animal. In particular, the terms refer to a canine. The terms also include humans, domestic animals including horses, cows, sheep, poultry, fish, pigs, cats, and zoo animals.

“Pluripotent” refers to cells which retain the developmental potential to differentiate into a variety of cell lineages including the germ line.

“Canine embryonic stem cell phenotype” is used to describe cells which are undifferentiated and which are visually distinguished from other adult cells of canines.

“Cell line” refers to cultured cells that can be passaged at least one time without terminating. The invention contemplates cell lines that can be passaged at least 1, 2, 5, 10, 15, 20, 30, 40, 50, 60, 80, 100, and 200 times.

“Effective amount” refers to concentrations of components such as cells, preparations, or compositions effective for producing an intended result including treating a disease or condition with cells, preparations, and compositions of the invention, or for effecting a transplantation of cells within a subject to be treated.

The terms “administering” or “administration” refers to the process by which cells, preparations, or compositions of the invention are delivered to a subject for treatment purposes. Cells, preparations, or compositions may be administered a number of ways including parenteral (e.g. intravenous and intraarterial as well as other appropriate parenteral routes), oral, subcutaneous, inhalation, or transdermal. Cells, preparations, and compositions of the invention are administered in accordance with good medical practices taking into account the subject's clinical condition, the site and method of administration, dosage, patient age, sex, body weight, and other factors known to physicians.

“Transplanting”, “transplantation”, “grafting” and “graft” are used to describe the process by which cells, preparations, and compositions of the invention are delivered to the site within the subject where the cells are intended to exhibit a favorable effect, such as repairing damage to a subject's tissues, treating a disease, injury or trauma, or genetic damage or environmental insult to an organ or tissue caused by, for example an accident or other activity. Cells, preparations, and compositions may also be delivered in a remote area of the body by any mode of administration relying on cellular migration to the appropriate area in the body to effect transplantation.

“Enriched” refers to a population of cells or a method which is at least 20+%, 30+%, 40+%, 50+%, 60+%, 70+%, 80+%, 85+%, 90+%, or 95+% effective, more preferably at least 98+% effective, most preferably 99+%/o effective. Therefore, a method that enriches for a given cell population, enriches at least about 20+%, 30+%, 40+%, 50+%, 60+%, 70+%, 80%, 85%, 90%, or 95% of the targeted cell population, most preferably at least about 98% of the cell population, most preferably about 99% of the cell population.

“Isolated” or “purified” refers to altered “by the hand of man” from the natural state i.e. anything that occurs in nature is defined as isolated when it has been removed from its original environment, or both. In an aspect, a population or composition of cells is substantially free of cells and materials with which it may be associated in nature. By substantially free or substantially purified is meant at least 50% of the population are the target cells, preferably at least 70%, more preferably-at least 80%, and even more preferably at least 90% are free of other cells. Purity of a population or composition of cells can be assessed by appropriate methods that are well known in the art.

“Gene therapy” refers to the transfer of new genetic information into cells for the therapeutic treatment of diseases or disorders. A foreign gene is transferred into a cell that proliferates to introduce the transferred gene throughout the cell population. Therefore, cells and compositions of the invention may be the target of gene transfer, since they may produce various lineages that will potentially express the foreign gene.

The term “embryo” as used herein refers to a developing cell mass that has not implanted into the uterine membrane of a maternal host. The term may refer to a fertilized oocyte, a pre-blastocyst stage developing cell mass, a blastocyst, and/or any other developing cell mass that is at a stage of development prior to implantation. Cells, cell lines, and cell preparations of the invention may be isolated from and/or arise from an embryo. An embryo can correspond to multiple stages of cell development The invention preferably contemplates an early stage embryo in particular, an embryo at a morula to expanded blastocyst stage.

“Morula” refers to the structure during embryonic development comprising 8 or more cells.

The term “blastocyst” used herein refers to the structure during early embryonic development comprising an inner cluster of cells, the inner cell mass (ICM), which gives rise to the embryo, and an outer layer, the trophectoderm, which gives rise to extra-embryonic tissues. In particular, cells from the ICM of an early or expanded blasotocyst may be used in the present invention. In a preferred embodiment, cells from a blastocyst obtained 9-14 days, more preferably 10-11 days, post ovulation are utilized in the invention.

Stem Cells and Cell Lines

The present invention provides cells exhibiting a canine embryonic stem cell phenotype, and a cell line comprising cells exhibiting a canine embryonic stem cell phenotype.

In an embodiment, the present invention relates to a pluripotent canine stem cell line. In another embodiment, the invention relates to a purified preparation comprising, or enriched for, canine embryonic stem cells that are capable of indefinite proliferation in vitro in an undifferentiated state.

Proliferation in vivo may include cultivation of the stem cells for prolonged periods where the cells are substantially maintained in an undifferentiated state. The undifferentiated cells may be capable of maintaining an undifferentiated state when cultured in the presence of a feeder layer. In a preferred aspect the feeder layer does not induce extraembryonic differentiation or cell death.

A preparation of canine embryonic stem cells of the invention may also be characterized by being immunoreactive with markers for canine embryonic stem cells. In an embodiment, the stem cells express genetic markers of canine embryonic stem cells, including but not limited to Oct-4, SSEA-4, TRA-1-60, and alkaline phosphatase.

The canine embryonic stem cells of the invention may be characterized as distinct from embryonic stem cells from other species. In particular, canine embryonic stem cells may be characterized as more closely resembling human than murine embryonic stem cells in their morphology, expression of cell surface antigens, growth rates, and passage requirements.

The canine ES cells of the invention preferably have the potential to differentiate in vitro when subjected to differentiating conditions. Most preferably the stem cells have the capacity to differentiate in vitro into derivatives of the three embryonic germ layers. The ability of the canine embryonic stem cells to differentiate in vitro into a variety of cell types including the ability to differentiate into embryonic and more highly differentiated cell types, may be tested by methods known in the art. For example, to induce differentiation in monolayer cultures, cells may be cultured without passage onto a fresh feeder layer. Differentiation may be induced in suspension culture by passing the cells onto a gelatinized plate to eliminate possible contamination by fibroblasts.

The invention therefore also relates to a purified canine embryonic stem cell preparation of the invention (preferably cultured in vitro) induced to differentiate into cells of various lineages. A differentiated cell preparation is characterized by expression of genetic markers of various cell lineages In an embodiment, the invention provides cells differentiated in vitro from an undifferentiated canine embryonic stem cell. In addition, a committed progenitor cell capable of giving rise to a mature somatic cell is provided. Preferably, undifferentiated cells are capable of differentiating into extraembryonic and embryonic lineages under differentiating conditions. In particular, the cells of the invention are capable of differentiating into cells derived from mesoderm, endoderm, and ectoderm germ layers when the cells are injected into an immunocompromised host.

A cell preparation of the invention may be derived from or comprised of cells that have been genetically modified either in nature or by genetic engineering techniques in vivo or in vitro.

Cell preparations or cell lines of the invention can be modified by introducing mutations into genes in the cells or by introducing transgenes into the cells. Insertion or deletion mutations may be introduced in a cell using standard techniques. A transgene may be introduced into cells via conventional techniques such as calcium phosphate or calcium chloride co-precipitation, DEAE-dextran-mediated transfection, lipofection, electroporation, or microinjection. Suitable methods for transforming and transfecting cells can be found in Sambrook et al. (Molecular Cloning: A Laboratory Manual, 2nd Edition, Cold Spring Harbor Laboratory press (1989)), and other laboratory textbooks. By way of example, a transgene may be introduced into cells using an appropriate expression vector including but not limited to cosmids, plasmids, or modified viruses (e.g. replication defective retroviruses—including lenti- and onco-retrovial vectors, adenoviruses and adeno-associated viruses). Transfection is easily and efficiently obtained using standard methods including culturing the cells on a monolayer of virus-producing cells (Van der Putten, Proc Natl Acad Sci USA. 1985 September; 82(18):6148-52; Stewart et al. (1987) EMBO J. 6:383-388).

A gene encoding a selectable marker may be integrated into cells of a cell preparation of the invention. For example, a gene encoding a protein such as β-galactosidase, chloramphenicol acetyltransferase, firefly luciferase, or a fluorescent protein marker may be integrated into the cells. Examples of fluorescent protein markers are the Green Fluorescent Protein (GFP), and variants thereof.

Method of Producing Stem Cells

The invention relates to a method for obtaining purified canine embryonic cells comprising the step of culturing ICM cells from a canine embryo under conditions that promote proliferation of undifferentiated cells. In an embodiment, the cells are cultured in the presence of a feeder layer (e.g. a fibroblast layer or a medium conditioned by fibroblasts), and one or more proliferation agent.

A method for obtaining canine embryonic stem cells of the invention may additionally comprise expanding or maintaining canine embryonic stem cells, and/or inducing differentiation of the stem cells, by for example, removing the feeder layer.

In an aspect the invention provides a method of obtaining cells exhibiting a canine embryonic stem cell phenotype. Cells exhibiting a canine embryonic stem cell phenotype may be isolated by (a) obtaining a canine embryo; (b) culturing inner cell mass (ICM) cells from the canine embryo under conditions which promote proliferation of undifferentiated stem cells; and (c) recovering stem cells. The conditions that promote proliferation of undifferentiated stem cells (i.e. prevent differentiation of stem cells) differ from the requirements for other species.

In an embodiment of the invention, a method of producing cells exhibiting a canine embryonic stem cell phenotype is provided comprising: (a) obtaining a canine embryo at a morula to expanded blastocyst stage; (b) removing inner cell mass (ICM) cells of the blastocyst; (c) culturing ICM cells in the presence of a feeder layer to promote proliferation of undifferentiated stem cells; and (c) recovering stem cells.

In an aspect of the invention there is provided a method of preparing a preparation enriched for undifferentiated canine embryonic stem cells comprising:
(a) obtaining a fertilized canine embryo;
(b) removing inner cell mass (ICM) cells from the embryo;
(c) culturing ICM cells under conditions which do not induce differentiation and promote proliferation of undifferentiated cells; and
(d) recovering stem cells.

In an embodiment of the invention, a method for obtaining canine embryonic stem cells is provided comprising:
(a) growing embryos from canines in the presence of a feeder layer;
(b) removing ICM cells of the embryos either after spontaneous hatching or after mechanical removal of the zona pellucida;
(c) growing the cells in the presence of a feeder layer;
(d) selecting stem cell colonies by morphological characteristics; and
(e) culturing the selected stem cells.

In an embodiment of the invention, the method comprises obtaining a canine embryo, culturing the embryo in the presence of a feeder layer and proliferation agents, removing a blastocyst outgrowth and transferring the outgrowth to a fresh feeder layer. After establishment of the culture of undifferentiated cells, undifferentiated ES colonies are selected, dissociated by mechanical manipulation or enzymatic digestion, and transferred to fresh cultures for propagation. The invention also contemplates cell preparations or lines derived at all stages of development under the same culture conditions.

The method may further comprise passaging the selected stem cells onto fresh tissue culture growth medium at intervals to prevent differentiation of the cells and to maintain a cell line in culture. Cell passaging may involve the steps of (1) releasing cells from a feeder layer and disassociation of these cells, and (2) placing the cells in media suitable for further cell proliferation. In an embodiment, cells are passaged by releasing cells from a surface using an enzymatic treatment. Cells that are released can then be diluted and transferred to fresh culture medium.

Canine embryos may be derived or isolated from any canine species. Canine species may include purebred species and species used as disease models or associated with congenital, single or multigene defects or disorders including hip dyspasia, and congenital heart defects. Suitable species include but not limited to a beagle, Doberman Pinscher, Ibizan Hound, Samoyed, Saluki, Maltese, Leonburger, and poodle. The canine embryos are harvested to provide maximum recovery and in vitro maturation and hatching of embryos. In an embodiment, the embryos are harvested after insemination or post ovulation.

Mutant or transgenic blastocysts may be used to prepare a cell preparation or cell line of the invention. Cells used to prepare a cell preparation or cell line of the invention can be engineered to contain a selectable marker or they may be genetically altered using techniques well known in the art.

A canine embryo (e.g. morula or bastocysts) used in a method of the invention may be maintained in culture under conditions permitting expansion of canine embryonic stem cells. Embryos may be cultured in the presence of a feeder layer. The feeder layer may be a confluent fibroblast layer, including primary mouse embryonic fibroblast (EMFI) cells or canine embryonic fibroblast like-cells. Embryonic fibroblasts may be obtained from 12 day old fetuses from outbred mice, but other strains may be used as an alternative. STO cells (i.e. a permanent line of irradiated mouse fibroblasts) can also be used as a feeder layer. The feeder layer may also comprise medium conditioned by primary embryonic fibroblast cells.

The conditions which promote proliferation of undifferentiated stem cells may involve culturing the cells in the presence of one or more proliferation agents including growth factors, chemicals or cytokines. The proliferation agents may be canine or human in origin, or may be derived from other mammalian species active on canine cells. The following are representative examples of proliferation agents which may be employed in the present invention: all members of the fibroblast growth factor (FGF) family including FGF-4 and FGF-2, epidermal growth factor (EGF), stem cell factor (SCF), thrombopoietin (TPO), FLT-3 ligand, neural growth factor (NGF), VEGF, Granulocyte-Macrophage Growth Factor (GM-CSF), HGF, Hox family, Notch, leukemia inhibitor factor (LIF), cardiotrophin 1 (CT-1), ciliary neurotrophic factor (CNTF), oncostatin M (OSM), and any member of the interleukin (IL) family, including IL-6, IL-11, and IL-12. Proliferation agents may be used in combination with equal molar or greater amounts of a glycosaminoglycan such as heparin sulfate.

Proliferation agents may be commercially available or can be produced by recombinant DNA techniques and purified to various degrees. For example, growth factors are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). Some proliferation agents may be purified from culture media of cell lines by standard biochemical techniques. Thus, it is intended that molecules having similar biological activity as wild-type or purified proliferation agents (e.g., recombinantly produced or mutants thereof) are intended to be used within the spirit and scope of the invention.

An effective amount of a proliferation agent is used in the culture medium. The proliferation agents are typically applied at sufficient intervals to maintain high proliferation levels and maintenance of a stem cell phenotype.

The culture medium used in the methods of the invention may comprise any medium that supports embryonic stem cells. The medium may be conditioned medium, non-conditioned medium, or embryonic stem cell medium. Examples of suitable conditioned medium include IMDM, DMEM, or αMEM, conditioned with embryonic fibroblast cells (e.g. canine embryonic fibroblast cells, human embryonic fibroblast cells or mouse embryonic fibroblast cells), or equivalent medium. Examples of suitable non-conditioned medium include Iscove's Modified Delbecco's Medium (IMDM), DMEM, or αMEM, or equivalent medium. The culture medium may comprise serum (e.g. canine serum, bovine serum, fetal bovine serum, calf bovine serum, horse serum, human serum, or an artificial serum substitute [e.g. 1% bovine serum albumin, 10 μg/ml bovine pancreatic insulin, 200 μg/ml human transferrin, 10⁻⁴M β-mercaptoethanol, 2 mM L-glutamine and 40 μg/ml LDL (Low Density Lipoproteins)], or it may be serum free. Preferably batch-tested serums are used.

In an embodiment, the culture medium is serum free to provide cells that are free of serum proteins or biomolecules that may bind to the surface of the cells. Cells cultured in such conditions may provide somatic cells that have potential exposed novel antigenic sites. Such cells may be useful as immunogens or tolerizing agents for immune suppression. Thus, the invention provides a cellular composition or mitotic or differentiated cells therefrom that are isolated and maintained in serum-free media.

In a preferred embodiment, the culture medium used for growth of embryonic stem cells includes KO DMEM medium, preferably supplemented with serum (e.g. canine serum or fetal bovine serum).

Embryos may be hatched spontaneously or manipulated mechanically to support hatching. The zona pellucida surrounding the ICM may be removed using chemical (e.g. pronase, acid Tyrodes solution) or mechanical (e.g. needle, blade, laser dissection) methods. Preferably mechanical methods are employed.

A method of the invention may involve treating the canine embryo to dislodge the trophectoderm of the embryo or portion thereof. Methods suitable for removing the trophectoderm include mechanical methods and immuno-surgery. The embryo (or blastocyst devoid of zona pellucida) may be treated with antibody or antiserum specific for trophectoderm epitopes and/or with complement.

Outgrowths or inner cell mass cells comprising ES-like cells may be removed and cultured in the presence of a feeder layer as described herein. The cells may be cultured for a sufficient period of time to establish the undifferentiated stem cells.

Once established the undifferentiated stem cells may be propagated, expanded, and maintained. Thus, a method for preparing canine embryonic stem cells may further include removing the stem cells to another feeder layer and culturing the stem cells for a period sufficient to obtain proliferation of an enriched preparation of morphologically undifferentiated stem cells. In order to expand and maintain the undifferentiated cells, cultured stem cells may be dissociated from the culture (e.g. using enzymatic or mechanical means) and cultured on fresh media. Cells may be regularly sub-cultured (e.g. every 2-7 days)

A method of the invention may further comprise preserving the canine embryonic stem cells or cell lines by preservation methods such as cryopreservation. Examples of suitable cryopreservation methods are those that are highly efficient for use with embryos such as vitrification, in particular the Open Pulled Straw (OPS) vitrification method.

A method of the invention may still further comprise inducing differentiation of the canine embryonic stem cells as described herein. The method may involve culturing the stem cells under conditions that promote differentiation (e.g. cell or tissue-specific differentiation). The method may facilitate the derivation of committed lineage progenitor cells which are no longer pluripotent but may give rise to cells of a variety of lineages.

Applications

Cells from the cell preparations may be introduced into a blastocyst or aggregated with an early stage embryo to produce chimeric conceptuses. A chimeric conceptus may be allowed to grow to term, or sacrificed during gestation to observe the contribution of the stem cells. The conceptuses can be engineered to carry selectable markers or genetic alterations. Cell lines can be derived from the chimeric conceptuses. Therefore, the invention further provides a chimeric conceptus derived from a purified preparation of the invention.

Cells of the invention may be used to repopulate an embryo of the same species thus giving rise to a chimeric animal, particularly a chimeric animal in which some or all of the germ cells are derived from the cultured cells. The embryonic stem cells may have been genetically modified or selected for genetic modification in culture.

The invention can provide for the derivation of canine embryonic stem cells from embryos carrying a particular genetic background or specific mutations. For example, the embryos can be derived from high-pedigree canines.

The cells, cell lines, cell preparations, chimeric conceptuses, embryos and chimeric animals of the invention may be used to screen for potential therapeutics that modulate development or activity e.g. proliferation. In particular, the cell preparations and chimeric embryos may be subjected to a test substance, and the effect of the test substance may be compared to a control (e.g. in the absence of the substance) to determine if the test substance modulates development or activity. Selected substances may be useful in regulating canine embryonic stem cells or progeny thereof in vivo and they may be used to treat various conditions requiring regulation of such cells.

The cells and cell preparations of the invention may be used to prepare model systems of disease or conditions. Canines develop similar diseases as humans and the clinical presentations are similar. Thus, canine models offer very useful models for studying disease and identifying potential therapeutics. Canine models of human diseases can be created including but not limited to models for glycogen storage disease, muscular dystrophy, haemophilia, narcolepsy, thrombasthenia, Von Willebrand Disease, osteogenesis, nephritis, retinal atrophy, severe combined immunodeficiency disease, hematopoietic and autoimmune disorders, cancer, heart diseases, motor neuron diseases, and degenerative bone and joint diseases, and atherosclerosis.

Canines provide a powerful preclinical large animal model in biomedical research, which historically has been used successfully to move novel treatment modalities into the clinic (reviewed by Ostrander et al (3)). Breeding programs for the generation of canines with distinctive phenotypes have led to the production of closed breeding populations characterized by more than 400 inherited disorders. Autosomal recessive and complex traits represent the largest proportion of canine diseases, some of which include hematopoietic and autoimmune disorders, cancer, heart diseases, motor neuron diseases, and degenerative bone and joint diseases. These naturally occurring canine diseases provide powerful models for genetic mapping and the assessment of the pathophysiology and novel treatments of homologous diseases in humans. Canines share many biochemical and physiologic characteristics with humans and thus they more accurately resemble human diseases than do their rodent counterparts. Their short generation time and long life span make them ideal for studying the lifetime effects of medical manipulations. Canines are more readily available, incur lower costs, are more disease-free and easier to work with than nonhuman primates. Compared with mice, the large size of canines is amenable to serial blood and tissue sampling and continuous intravenous infusions. Since canines closely approximate humans in body weights, blood volumes, and issues of tissue typing and clinical management, they have been instrumental in the development of human bone marrow transplantation and gene therapy protocols (31-42). Large canine breeds have also made valuable contributions to the development of treatment modalities for cardiovascular (43) and orthopaedic diseases (44, 45). The availability of canine ES cells as described herein facilitates the development of ES cell-based therapies for the treatment of inherited and acquired human diseases.

The cells, cell preparations or cell lines of the invention can be used to produce growth factors and hormones. The cell preparations or cell lines of the invention can also be used to produce therapeutics.

The canine embryonic stem cells of the invention may be induced to differentiate into cells of a variety of lineages, preferably cells that exhibit morphological, physiological, functional, and/or immunological features of somatic and germ cells. Cells from a differentiated cell preparation may be characterized by expression of genetic markers from a variety of cell lineages (e.g. markers for muscle, neural, adipocyte, osteoclast, osteoblast, endothelial, hematopoietic, astrocytes, pancreatic cells, retinal cells, renal cells, connective tissue cells, and hepatocytes), or physiological, immunological or functional characteristics of cells of a variety of lineages. For example, cells can be screened for expression of tissue specific markers such as Myo-D (muscle), FLK-1 (endothelial), glial fibrillary acidic protein (astrocytes), glucagon (alpha-α cells), insulin (islet-β cells), somatostatin (islet-δ), pancreatic polypeptide (islet-PP cells), cytokeratins (CK), mucin MUC1, carbonic anyhydrase II, and carbohydrate antigen 19.1 (ductal cells), and NESTIN (neural).

Differentiated cells can be used to prepare a cDNA library relatively uncontaminated with cDNA preferentially expressed in cells from other lineages, and they can be used to prepare antibodies that are specific for particular markers of somatic cells.

After differentiation of the cells into selected somatic cells as described herein, the cells may be separated to obtain a population of cells largely consisting of somatic cells. This may be accomplished by positive selection of somatic cells using antibodies to identify tissue specific cell surface markers or negative selection using ES cell specific markers.

A cell preparation or cellular composition of the invention may be genetically engineered in such a manner that they or cells derived therefrom produce, in vitro or in vivo, polypeptides, hormones and proteins not normally produced in the cells in biologically significant amounts, or produced in small amounts but in situations in which regulatory expression would lead to a therapeutic benefit For example, the cells could be engineered with a gene that expresses a molecule that specifically inhibits bone resorption, but does not otherwise interfere with osteoclasts binding to bone, or the cells could be engineered with a gene that expresses insulin at levels compatible with normal injected doses. Alternatively the cells could be modified such that a protein normally expressed will be expressed at much lower levels. These products would then be secreted into the surrounding media or purified from the cells. The cells formed in this way can serve as continuous short term or long term production systems of the expressed substance.

Thus, in accordance with this aspect of the invention, cells of the invention can be modified with genetic material of interest. The modified cells can be cultured in vitro under suitable conditions so that they differentiate into cells of specific lineages. The cells are able to express the product of the gene expression or secrete the expression product. These modified cells can be administered to a target tissue where the expressed product will have a beneficial effect.

In a further embodiment, the transduced cells of the invention can be induced in vivo to differentiate into cells of specific lineages that will express the gene product For example, the transduced cells may be administered to induce production of cells of specific lineages having the transduced gene. The cells may be administered in admixture with each other or separately and may be delivered to a targeted area. The cells can be introduced intravenously and home to the targeted area. Alternatively, the cells may be used alone and caused to differentiate in vivo.

Thus, genes can be introduced into cells which are then injected into a recipient where the expression of the gene will have a therapeutic effect. For example, osteoclasts may be genetically engineered to have reduced activity in vivo. Appropriate genes would include those that play a role in the regulation of osteoporosis, in areas such as serum calcium responsiveness, estrogen secretion and bone resorption. An insulin gene may be introduced into blood stem cells to provide a constant therapeutic dose of insulin in the bone marrow and peripheral blood.

The technology may be used to produce additional copies of essential genes to allow augmented expression by cells of certain gene products in vivo. These genes can be, for example, hormones, matrix proteins, cell membrane proteins, cytokines, adhesion molecules, or “rebuilding” proteins important in tissue repair.

The cell preparations and compositions of the invention can be used in a variety of methods (e.g. transplantation) and they have numerous uses in the field of medicine. They may be used for the replacement of body tissues, organs, components or structures which are missing or damaged due to trauma, age, metabolic or toxic injury, disease, idiopathic loss, or any other cause. In particular, they may have application in the study, prevention, and treatment of conditions such as hemophilia, muscular dystrophy, MPS-1, glycogen storage disease, narcolepsy, thrombasthenia, Von Willebrand Disease, osteogenesis, nephritis, retinal atrophy, severe combined immunodeficiency disease, hematopoietic and autoimmune disorders, cancer, heart diseases, motor neuron diseases, degenerative bone and joint diseases, and atherosclerosis.

Transplantation or grafting, as used herein, can include the steps of isolating a cell preparation according to the invention and transferring cells in the preparation into a mammal or a patient. Transplantation can involve transferring the cells into a mammal or a patient by injection of a cell suspension into the mammal or patient, surgical implantation of a cell mass into a tissue or organ of the mammal or patient, or perfusion of a tissue or organ with a cell suspension. The route of transferring the cells may be determined by the requirement for the cells to reside in a particular tissue or organ and by the ability of the cells to find and be retained by the desired target tissue or organ. Where the transplanted cells are to reside in a particular location, they can be surgically placed into a tissue or organ or simply injected into the bloodstream if the cells have the capability to migrate to the desired target organ.

The invention may be used for autografting (cells from an individual are used in the same individual), allografting cells (cells from one individual are used in another individual) and xenografting (transplantation from one species to another). Thus, the cells, cell preparations and cellular compositions of the invention may be used in autologous or allogenic transplantation procedures to improve a cell deficit or to repair tissue.

In an aspect of the invention, the newly created cells, cell lines, and preparations, can be used in both cell therapies and gene therapies aimed at alleviating disorders and diseases involving the cells or progeny thereof The invention obviates the need for human tissue to be used in various medical and research applications.

The cell therapy approach involves the use of transplantation of the newly created cells, cell lines, or preparations or cells differentiated therefrom, as a treatment for injuries and diseases. The steps in this application include: (a) producing cells or a cell line of the invention, or differentiating cells therefrom, as described herein; and (b) allowing the cells to form functional connections either before or after a step involving transplantation of the cells. The gene therapy approach also involves cellular compositions comprising cells of the invention transfected with an appropriate vector containing a cDNA for a desired protein, followed by a step where the modified cells are transplanted.

In either a cell or gene therapy approach, therefore, cells of the present invention, or cells or tissues differentiated from the cells can be transplanted in, or grafted to, a patient in need. Thus, the cells or differentiated cells therefrom can be used to replace the cells in a patient in a cell therapy approach, useful in the treatment of tissue injury, and diseases. These cells can be also used as vehicles for the delivery of specific gene products to a patient One example of how these newly created cells or cells differentiated therefrom can be used in a gene therapy method is in treating the effects of Parkinson's disease. For example, tyrosine hydrolase, a key enzyme in dopamine synthesis, may be delivered to a subject via the transplantation of cells of the invention that are capable of differentiating into neuronal cells, or transplantation of neuronal cells differentiated from the cells, which have been transfected with a vector suitable for the expression of tyrosine hydrolase.

The invention also provides a method of treating a subject with a condition involving a somatic cell of the invention comprising transferring a cell of the invention into the subject, wherein the cell differentiates into the somatic cell.

The invention also contemplates a pharmaceutical composition comprising cells, a cell preparation, or cell line of the invention, and a pharmaceutically acceptable carrier, excipient, or diluent. The pharmaceutical compositions herein can be prepared by per se known methods for the preparation of pharmaceutically acceptable compositions which can be administered to subjects, such that an effective amount of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the cells, cell preparations, or cell lines in association with one or more pharmaceutically acceptable vehicles or diluents, and contained in buffered solutions with a suitable pH and iso-osmotic with the physiological fluids.

Still another aspect of the invention is a kit for producing cells of the invention. The kit includes the reagents for a method of the present invention for producing cells of the invention.

In an aspect, cells, cell preparations, and cellular compositions disclosed herein can be used for toxicity testing for drug development testing. Toxicity testing may be conducted by culturing cells, cell preparations, and cell lines or cells differentiated therefrom in a suitable medium and introducing a substance, such as a pharmaceutical or chemical, to the culture. The cells or differentiated cells are examined to determine if the substance has had an adverse effect on the culture. Drug development testing may be done by developing derivative cell lines which may be used to test the efficacy of new drugs. Affinity assays for new drugs may also be developed from the cells, differentiated cells, or cell lines.

Using a method of the invention it is possible to identify drugs that are potentially toxic to canine embryonic stem cells.

The cellular compositions of the invention may be used to screen for potential therapeutics that modulate development or activity of cells of the invention. In particular, the cells of the invention may be subjected to a test substance, and the effect of the test substance may be compared to a control (e.g. in the absence of the substance) to determine if the test substance modulates development or activity of the cells or cells differentiated therefrom.

In an aspect of the invention a method is provided for using cells of the invention to assay the activity of a test substance comprising the steps of:
(a) exposing the cells to a test substance; and
(b) detecting the presence or absence of an effect of the test substance on the survival of the cells or on a morphological, functional, or physiological characteristic and/or molecular biological property of the cells, whereby an effect altering cell survival, a morphological, functional, or physiological characteristic and/or a molecular biological property of the cells indicates the activity of the test substance.

In another aspect a method is provided for using cells of the invention to screen a potential new drug to treat a disorder involving the cells comprising the steps of:
(a) exposing the cells to a potential new drug, and
(b) detecting the presence or absence of an effect of the potential new drug on the survival of the cells or on a morphological, functional, or physiological characteristic and/or molecular biological property of said cells, whereby an effect altering cell survival, a morphological, functional, or physiological characteristic and/or a molecular biological property of the cells indicates the activity of the potential new drug.

The invention also relates to the use of cells, cell lines, cell preparations, and compositions in drug discovery. The invention provides methods for drug development using the cells, cell preparations, and cellular compositions of the invention. Cells, cell preparations, cell lines, and compositions of the invention may comprise cells that secrete novel or known biological molecules or components. In particular, culturing in the absence of serum may provide cells that have minimal interference from serum molecules and thus, may be more physiologically and topologically accurate. Therefore, proteins secreted by cells described herein may be used as targets for drug development. In one embodiment, drugs can be made to target specific proteins on cells of the invention. Binding of the drug may promote differentiation of cells into cells of specific lineages. In another embodiment, drugs specific for regulatory proteins of somatic cells may be used to arrest growth of a particular type of cell. Any of the proteins can be used as targets to develop antibody, protein, antisense, aptamer, ribozymes, or small molecule drugs.

Agents, test substances, or drugs identified in accordance with a method of the invention or used in a method of the invention include but are not limited to proteins, peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)2, and Fab expression library fragments, and epitope-binding fragments thereof)], nucleic acids, ribozymes, carbohydrates, and small organic or inorganic molecules. An agent, substance or drug may be an endogenous physiological compound or it may be a natural or synthetic compound.

The cells, cell preparations, and cell lines disclosed herein can be used in various bioassays. In an embodiment, the cells are used to determine which biological factors are required for proliferation or differentiation. By using cells of the invention in a stepwise fashion in combination with different biological compounds (such as hormones, specific growth factors, etc.), one or more specific biological compounds can be found to induce differentiation to somatic cells. Other uses in a bioassay for the cells are differential display (i.e. mRNA differential display) and protein-protein interactions using secreted proteins from the cells. Protein-protein interactions can be determined with techniques such as a yeast two-hybrid system. Proteins from cells, cell preparations and cellular compositions of the invention can be used to identify other unknown proteins or other cell types that interact with the cells. These unknown proteins may be one or more of the following: growth factors, hormones, enzymes, transcription factors, translational factors, and tumor suppressors. Bioassays involving cells, cell preparations, and cell lines of the invention, and the protein-protein interactions these cells form and the effects of protein-protein or cell-cell contact may be used to determine how surrounding tissue contributes to proliferation or differentiation of cells of various lineages.

In an aspect of the invention cells of the invention may be used to repair cell or tissue injury. They may also be used in the treatment of genetic defects that result in nonfunctional cells. The stem cells of the invention may be transplanted directly to the site of defective cells in order to rescue the defect or delivered via the blood stream by injecting the cells into the vein. In addition, gene therapy vectors may be integrated into the stem cells followed by engraftment of these engineered cells to their target tissues. The introduction of gene therapy vectors requires cell proliferation. The successful long term engraftment of the cells to the target tissue requires they maintain a stem cell characteristic.

The cells, cell preparations, and cell lines of the invention may be used as immunogens that are administered to a heterologous recipient. Administration of cells obtained in accordance with the invention may be accomplished by various methods. Methods of administering cells as immunogens to a heterologous recipient include without limitation immunization, administration to a membrane by direct contact (e.g. by swabbing or scratch apparatus), administration to mucous membranes (e.g. by aerosol), and oral administration. Immunization may be passive or active and may occur via different routes including intraperitoneal injection, intradermal injection, and local injection. The route and schedule of immunization are in accordance with generally established conventional methods for antibody stimulation and production. Mammalian subjects and antibody producing cells therefrom may be manipulated to serve as the basis for production of mammalian hybridoma cell lines.

In an aspect the invention provides a culture system from which genes, proteins, and other metabolites involved in proliferation or differentiation of cells of various lineages can be identified and isolated. The cells in a culture system of the invention may be compared with other cells (e.g. differentiated cells) to determine the mechanisms and compounds that stimulate production of cells of various lineages.

The cells of the invention can be used to screen for genes expressed in or essential for differentiation of canine embryonic stem cells. Screening methods that can be used include Representational Difference Analysis (RDA) or gene trapping with for example SA-lacZ. Gene trapping can be used to induce dominant mutations (e.g. by deleting particular domains of the gene product) that affect differentiation or activity of cells of the invention and allow the identification of genes expressed in or essential for differentiation of these cells.

Cell preparations of the invention comprising hematopoietic cells may be used for enhancing the immune and hematopoietic system of a subject The cell preparations will facilitate enhancement or reconstitution of the subject's immune and/or blood forming system.

In an aspect of the invention, the cells, cell lines, and cell preparations of the invention are used in the treatment of leukemia (e.g. acute myelogenous leukemia, chronic myelogenous leukemia), lymphomas (e.g. non-Hodgkin's lymphoma), neuroblastoma, testicular cancer, multiple myeloma, melanomas, breast cancer, solid tumors that have a stem cell etiology, or other cancers in which therapy results in the depletion of hematopoietic cells.

In another aspect of the invention, cells, cell lines, and compositions of the invention, with or without genetic modification to provide resistance to HIV, are used to treat subjects infected with HIV-1 that have undergone severe depletion of their hematopoietic cell compartment resulting in a state of immune deficiency.

Hematopoietic cells may also be transfected with a desired gene that can be used for treatment of genetic diseases. Hematopoietic cell-related genetic diseases can be treated by grafting with cells transfected with a gene that can make up for the deficiency or the abnormality of the gene causing the diseases. For example, a normal wild type gene that causes a disease such as β-thalassemia (Mediterranean anemia), sickle cell anemia, ADA deficiency, recombinase deficiency, recombinase regulatory gene deficiency and the like, can be transferred into the hematopoietic cells by homologous or random recombination and the cells can be grafted into a subject. Further, a preparation comprising normal hematopoietic cells free from abnormalities of genes (from a suitable donor) can be used for treatment.

Another application of gene therapy permits the use of a drug in a high concentration, which is normally considered to be dangerous, by providing drug resistance to normal hematopoietic cells by transferring a drug resistant gene into the cells. In particular, it is possible to carry out the treatment using an anticancer drug in high concentration by transferring a gene having drug resistance against the anticancer drug, e.g., a multiple drug resistant gene, into hematopoietic cells of the invention.

Diseases other than those relating to the hematopoietic system can be treated by using the hematopoietic cells of the invention in so far as the diseases relate to a deficiency of secretory proteins such as hormones, enzymes, cytokines, growth factors and the like. A deficient protein can be induced and expressed by transferring a gene encoding a target protein into the hematopoietic cells under the control of a suitable promoter. The expression of the protein can be controlled to obtain the same activity as that obtained by the natural expression in vivo.

It is also possible to insert a gene encoding a ribozyme, an antisense nucleic acid or the like or another suitable gene into the hematopoietic cells to control expression of a specific gene product in the cells or to inhibit susceptibility to diseases. For example, the hematopoietic cells can be subjected to gene modification to express an antisense nucleic acid or a ribozyme, which can prevent growth of hematic pathogens such as HIV, HTLV-I, HTLV-II and the like in hematopoietic cells.

The cells and cell preparations comprising hematopoietic cells of the invention can be introduced in a vertebrate, which is a recipient of cell grafting, by, for example, conventional intravenous administration.

The invention also relates to a method for conducting a regenerative medicine business, comprising: (a) a service for accepting and logging in samples from a client comprising cells of the invention; (b) a system for culturing cells dissociated from the samples; (c) a cell preservation system for preserving cells generated by the system in (b) for later retrieval on behalf of the client or a third party. The method may further comprise a billing system for billing the client or a medical insurance provider thereof.

The invention features a method for conducting a stem cell business comprising identifying agents which influence the proliferation, differentiation, or survival of cells of the invention. Examples of such agents are small molecules, antibodies, and extracellular proteins. Identified agents can be profiled and assessed for safety and efficacy in animals. In another aspect, the invention contemplates methods for influencing the proliferation, differentiation, or survival of cells of the invention by contacting the cells with an agent or agents identified by the foregoing method. The identified agents can be formulated as a pharmaceutical preparation, and manufactured, marketed, and distributed for sale.

In an embodiment, the invention provides a method for conducting a stem cell business comprising (a) identifying one or more agents which affect the proliferation, differentiation, function, or survival of cells of the invention; (b) conducting therapeutic profiling of agents identified in (a); or analogs thereof for efficacy and toxicity in animals; and (c) formulating a pharmaceutical composition including one or more agents identified in (b) as having an acceptable therapeutic profile. The method may further comprise the step of establishing a distribution system for distributing the pharmaceutical preparation for sale. The method may also comprise establishing a sales group for marketing the pharmaceutical preparation.

The invention also contemplates a method for conducting a drug discovery business comprising identifying factors that influence the proliferation, differentiation, function, or survival of cells of the invention, and licensing the rights for further development.

Having now described the invention, the same will be more readily understood through reference to the following examples which are provided by way of illustration, and are not intended to be limiting of the present invention.

### EXAMPLE 1

Generation Of Canine Embryonic Stem Cells For Use In Animal Models Of Human Disease

Pluripotent embryonic stem cells are undifferentiated cells that retain the ability to proliferate indefinitely and differentiate to cells of the three embryonic germ layers and their derivatives. This unlimited life span and wide developmental potential suggest that these cells have enormous potential for both basic research and clinical applications focused on regenerative medicine and tissue engineering. Herein is described the derivation of four canine embryonic stem (ES) cell lines. Preimplantation embryos collected on days 9-13 post-insemination were maintained on murine feeder layers under conditions used for the expansion of human ES cells. Canine ES cells have been maintained in an undifferentiated state and have undergone multiple in vitro passages in culture and multiple rounds of cryoperservation and thawing since their derivation. Similar to human ES cells, canine ES cells express Oct4, SSEA4, TRA-1-60 and alkaline phosphatase and do not express SSEA-1. Plating of ES cells at low density in the absence of fibroblasts resulted in their differentiation or death whereas low density seeding of ES cells onto a sparse feeder layer resulted in the formation of embryoid bodies. Undifferentiated ES cells may be maintained and expanded, and canine ES cells have been differentiated to endothelial cells and neuronal cells in vitro.

Materials and Methods

Mating and Embryo Collection

Fourteen female dogs of mixed breeding were used in this study. Nine to 13 days post-ovulation or 6 to 12 days after first mating, dogs were subjected to surgical procedure under general anesthesia. Both oviducts and the uterine horns were removed and each uterine horn was flushed using warm Dulbecco's Phosphate Buffered Saline (DPBS). After inspection embryos were transferred to CO₂ independent medium. On average, eight embryos were obtained per experiment (range: 0 to 16). A total of 59 embryos were collected throughout the study. No embryos were recovered from three dogs.

In Vitro Culture of Collected Embryos and Derivation of ES Cells

Embryos were cultured in Knock Out Dulbecco's Modified Eagle's Medium (KO DMEM) or in a 1:1 mixture of Dulbecco's Modified Eagle's Medium and Ham's nutrient mixture F-12 (DMEM/F12) (Invitrogen). Complete KO DMEM or DMEM/F12 media were supplemented with 0.1 mM β-mercaptoethanol (Sigma), 0.1 mM non-essential amino acids, 0.1 mM sodium pyruvate, penicillin (100 IU/ml), streptomycin (50 μg/ml) (Invitrogen), LIF (20 ng/ml) (Chemicon) and 15% FBS (Invitrogen). In some experiments, 15% of serum replacement media (SRM) (Invitrogen) was used in place of fetal bovine serum (FBS). Embryos were maintained at 37° C. or 38° C. in 5% CO₂.

Upon completion of optimization experiments, cultures were maintained in expansion medium prepared with Complete DMEM/F12 medium supplemented with 15% dog serum or FBS at 38° C. To prepare dog serum, 50 to 100 ml peripheral blood was obtained from dogs from which embryos had been harvested and centrifuged at 2000 rpm at room temperature for 30 min. Serum was collected after centrifugation, heat inactivated at 56° C. for 1 hour and sterilized using a 0.22 μm filter.

Mouse Embryo Fibroblasts (MEFs) or Canine Embryo Fibroblast-like cells (CEF-like) were used as feeder layers. MEFs were prepared by procedures outlined in a laboratory manual, “Manipulation of Mouse Embryo”, Cold Spring Harbor Laboratory Press-Second Edition. To establish CEF-like feeders, canine embryo-derived cells that underwent spontaneous differentiation and gave rise to fibroblast looking cells were cultured in DMEM/F12 supplemented media containing 15% FBS without LIF. Once confluent, cells were cultured under conditions identical to those described for the established of MEFs.

All feeders were inactivated by exposure to 10 μg/ml of mytomicin C or by γ-irradiation (4000 rads) as per the above mentioned laboratory manual.

Establishment and Maintenance of Canine ES Cell Lines

Embryos were cultured in expansion medium and allowed to hatch either spontaneously or manipulated mechanically to support hatching. Mechanical hatching was accomplished by cutting the zona pellucida surrounding the ICM with a fine scalpel blade. One or two openings were cut through the zona pellucida and trophectoderm while ensuring no damage to embryonic cells was incurred. In some cases, the zona pellucida was gently split and removed from the embryo using fine needles and the released ICM collected and plated onto fresh feeders. Five to 10 days after hatching, embryo-derived outgrowths were disaggregated into small pieces by mechanical cutting and gentle dissociation using a finely pulled glass pipette. The clusters of ES-like cells were transferred at high density to fresh MEFs and resulting colonies further sub-cultured every 2-4 days by mechanical manipulation with or without addition of 0.125% Dispase in Hanks' Balanced Salt Solution. Once established, ES cell lines were passaged every 3 to 7 days by exposure to 0.1% collagenase/dispase (Sigma) prepared in DMEM/F12 or 0.125% Dispase in Hanks' Balanced Salt Solution for 45-60 min followed by a brief exposure to 0.02% EDTA (Sigma). Cultures were maintained at 38° C. in 5% CO₂. Canine ES cultures were passaged at high density to maintain the undifferentiated phenotype. Cultures were examined daily and complete or half medium changes done on alternate days.

Cell lines were cryopreserved in cryomedium containing either DMEM/F12 supplemented with 10% DMSO and 20% dog serum or 90% FBS and 10% DMSO. Cells were cryopreserved under slow-cooling conditions, initially stored at −80° C. and subsequently transferred to liquid nitrogen. ES cells were recovered from cryopreservation by immersion of cryovials for 30-60 seconds in a 37° C. water bath. Cells were washed in DMED/F12 supplemented with 30% dog serum or FBS and spun at 1000 rpm prior to plating to remove DMSO. Cells were plated on irradiated MEFs in six-well dishes in complete DMEM/F12 medium supplemented with 15% dog serum or FBS and hLIF.

Cloning and Sequencing of Canine Oct4

In order to design primers specific for canine Oct4, human, mouse and bovine Oct4 homologous genes were obtained by BLAST search and aligned using Vector NTI 7.1 (InforMax, Inc. USA). The Oct4R1 primer was derived from the murine Oct2 sequence while the, Oct4S1 and Oct4A1 primers were designed based on the human Oct4 sequence. (Xu C, Inokuma M S, Denham J, Golds K, Kundu P, Gold J D, Carpenter M K. Feeder-free growth of undifferentiated human embryonic stem cells. Nature Biotechnology October 2001; 19(10):971-4, 2001)

| | |
|---|---|
| Oct4R1: ACTCGAACCACATCCTTCTCTAGC | [SEQ ID NO.:2] |
| Oct4S1: CTTGCTGCAGAAGTGGGTGGAGGAA | [SEQ ID NO.:3] |
| Oct4A1: CTGCAGTGTGGGTTTCGGGCA | [SEQ ID NO.:4] |

Total RNA was prepared from two early passage canine ES cell lines (ES1 and ES2), murine ES cells and murine TS cells using a Trizol kit (Invitrogen, USA). mRNA was extracted using dynal beads (Dynal A. S, Oslo, Norway). cDNA was generated with 100 ng of mRNA using oligo dT and reverse transcription using Superscript II reverse transcriptase (Invitrogen, USA). The canine Oct4 cDNA was amplified by PCR using primers, Oct4S1 and Oct4R1 for 45 cycles. The PCR products were diluted and used as template in a nested PCR reaction using primers, Oct4S1 and Oct4A1.

The PCR protocol consisted of a 3 minute denaturation at 94° C., and 45 cycles, each consisting of a 15 seconds denaturation phase at 94° C., a 30 second annealing period at 55° C. and a 1 minute extension time at 72° C. A final extension of 10 minutes at 72° C. was included. PCR products were cloned and sequenced to derive the canine Oct4 sequence.

Canine Oct4 RT-PCR of Canine ES-like Cells

The above derived canine Oct4 fragment was sequenced and the sequence used to design primers specific for canine Oct4.

| | |
|---|---|
| Dog-POU5F1-S1: TGACGACAACAAAAATCT | [SEQ ID NO.:5] |
| Dog-POU5F1-A1: CAGGCATGTGTTCTCCAG | [SEQ ID NO.:6] |

Oct4 expression was assessed by reverse transcriptase polymerase chain reaction (RT-PCR). cDNA from canine ES like-cells was prepared as described above. Oct4 cDNA was PCR amplified in reactions consisting of a 3 minute denaturation at 94° C., and 45 cycles, each consisting of denaturation for 30 seconds at 94° C., primer annealing at 50° C. for 30 seconds and extension at 72° C. for 30 seconds. A final extension for 7 minutes was at 72° C.

Immunofluorescence Labeling of Canine ES-Like Cells

Canine ES colonies were grown in 4-well slide chambers. Before staining, the medium was discarded and the cells were rinsed with PBS. Cells were fixed in cooled methanol at −20° C. After 10 minutes methanol was removed and the cell membrane was permeabilized with cooled acetone for 1 min at −20° C. Slides were washed twice with 4° C. PBS for 5 minutes, incubated 4 hours at 4° C. in 8% BSA to block cells and washed again.

Cells were incubated with primary antibody SSEA-1, SSEA-4 or TRA-1-60 (Chemicon International) at the concentration of 15 μg/ml in PBS containing 2% BSA for 2-4 hours or overnight and slides were washed three times for 5 minutes with PBS supplemented with 2% BSA. An anti-mouse IgG (Fab Specific) FITC labeled antibody (Sigma) was used as a secondary antibody (1:200 dilution). Secondary labeling was done for 20 minutes in PBS supplemented with 2% BSA and slides washed again as described above. Excess PBS was removed, slides mounted with mounting medium and covered with cover slips. Antibody labeling was detected by confocal microscopy and results recorded.

Analysis of Alkaline Phosphatase Activity

Expression of alkaline phosphatase (AP) in ES colonies was detected treating cells with BM Purple AP substrate (Roche) at 4° C. according to the manufacturer's protocols. Mouse ES cells and their differentiated progeny were used as positive and negative controls for AP activity.

In Vitro Formation of Embryoid Bodies (EBs)

ES cell lines were plated at low density in the absence of fibroblasts on gelatin-treated four-well tissue culture plates in DMEM/F12 supplemented with 15% canine serum, 2 mM glutamax, 0.1 mM β-mercaptoethanol and 1% nonessential amino acids. EBs were visible 24-48 hours after plating.

In Vitro Differentiation of Canine ES Cells to Endothelial Cells

Small clumps of cells obtained after routine passage or day 2-4 EBs were plated on Collagene IV (Sigma) treated tissue culture plates. Cells were cultured in alpha-MEM medium supplemented with 10% FBS, 0.1 mM β-mercaptoethanol, penicillin (100 IU/ml) and streptomycin (50 μg/ml). Visualization of endothelial cells was performed by direct immunoflourescence using Anti-CD31: RPE (PECAM-1, Serotec).

In Vitro Differentiation of Canine ES Cells to Neuronal Cells

Undifferentiated ES cells were plated into bacterial dish in ES cell medium without hLIF. One μM all-trans retinoic acid (RA) was added to the medium. On day 4 cell aggregates were replated on tissue culture slides in medium lacking RA. Half of the medium was removed and replaced with fresh media every 3-4 days and neuronal cell differentiation was assessed by morphological appearance.

In Vitro Differentiation of Canine ES Cells To Cardiomyocytes

Differentiation of ES cells was induced by removal of cells from MEF feeders followed by cultivation in suspension for the generation of three-dimensional differentiating embryoid bodies (EBs). EBs were transferred to tissue culture grade plates and after an additional incubation period, cardiomyocyte tissue within the EBs was identified by the presence of spontaneously contracting areas.

Results and Discussion

Optimization of Culture Conditions

To identify in vitro culture conditions that support the attachment and hatching of canine embryos, embryos harvested 9 to 13 days post ovulation were cultured under conditions that support the maintenance of human or mouse ES cells. The results of these studies are summarized in Table 1. A total of eight compact morula stage embryos were collected and plated on fresh MEFs. When cultured in DMEM/F12 supplemented with 15% FBS, two of two embryos attached and compact morula developed to early and expanded blastocysts. Initially the zona pellucida of harvested embryos was dense and thick. However, in vitro culture of embryos resulted in a thinning of the zona pellucida and expansion of the number of cells of the inner cell mass (ICM). After five to ten days in culture, blastocysts hatched and large outgrowths of embryo-derived IMC were detected within three to five days of hatching. Similar results were achieved with one of two embryos plated in KO DMEM supplemented with 15% FBS. Supplementation of DMEM/F12 media with serum replacement media (SRM) in place of FBS facilitated attachment of two of four compact morulae within 24 hr. However both embryos failed to develop beyond the compact morulae stage.

The ability of canine embryo derived fibroblast like cells (CEF-like cells) to provide feeder support with respect to embryo attachment and expansion was tested. Although, CEF-like cells enabled more rapid expansion and hatching of embryos, cells derived from the hatched embryos did not generate outgrowths with an ES cell appearance. Therefore, optimal maintenance and maturation of canine embryos occurred on MEFs in medium supplemented with either canine serum or batch-tested FCS under culture conditions similar to those used for the establishment of human ES cell lines.

Embryonic Developmental Stages and Expansion In Vitro

Collected embryos were classified based on post-ovulation date and developmental stage at the time of collection. Classification of the embryos collected used in this study are presented in Table 2. Four of the embryos were at the 16-cell morula stage, nine were compact morulae, four were early blastocysts, two were expanded blastocysts and six were contracted, hatching blastocysts with an expanded or broken zona pellucida. Canine embryos developed to the compact morula stage by day ten post-ovulation and by day 12-13 the majority of embryos had hatched. In addition, the size and developmental stage of embryos collected from the same or different females often varied with respect to developmental stage.

To further optimize the timing of embryo harvests resulting in the maximum number of embryo hatchings and in vitro expansion of ICM outgrowths, embryos were maintained in culture and examined daily. A summary of the results of in vitro embryo development are presented in Table 3. Thirteen of 13 embryos collected as compact morula stage and early/expanded blastocysts (days 10 and 11, respectively) were viable in in vitro culture conditions. Embryos attached within 24 to 48 hours of being plated in culture, and significant embryo expansion and increase in ICM cell numbers were observed following attachment. The spontaneous hatching and outgrowth formation was noted in 12 of the 13 compact Morula, early/expanded blastocyst embryos plated. In contrast, of the twelve 16-cell morula stage or hatching blastocyst stage (days 9 and 12 to 13, respectively) embryos plated six attached, with only three of these showing minimal expansion within the next few days. Eventually, these embryos decreased in size, showed signs of degeneration and did not survive the initial culture period. This finding could be due to suboptimal culture conditions required to support these stages of embryo development. In addition, it is possible that significant expansion of the ICM after blastocyst hatching did not occur in vitro due either to differentiation occurring in vivo prior to embryo harvest or to in vitro culture conditions. Thus these studies demonstrated that the optimal time for embryo harvest that assures maximum recovery and in vitro maturation of embryos was between days 10-11 after insemination.

Expansion of ICM and Establishment of ES Cell Lines

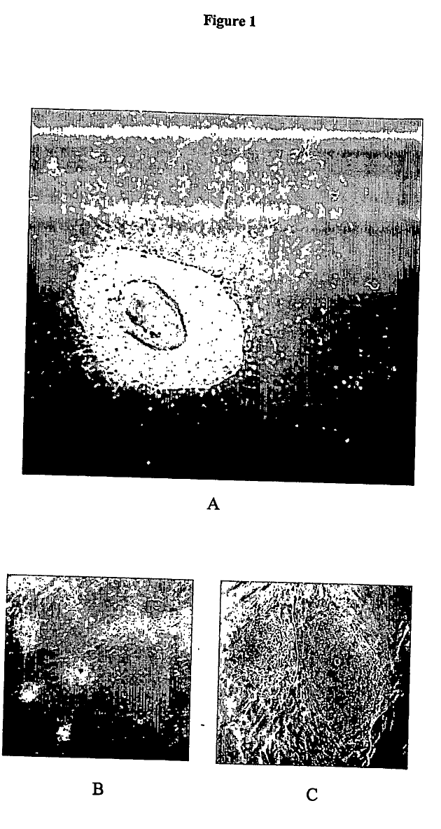

Of the 26 embryos collected 10 to 12 days post-ovulation, three were compact morulae, four were early blastocysts and 14 were expanded blastocysts. Five embryos were at a degenerated morula stage and although plated under optimal culture conditions did not demonstrate further in vitro maturation or expansion of the ICM. Embryos were maintained in culture and examined daily for attachment and expansion of ICM. As presented in Table 4, embryo hatching occurred either spontaneously or by mechanical manipulation. Zona pellucida from the first group of embryos, consisting mainly of expanded blastocysts, was mechanically cut open to facilitate the spontaneous release, outgrowth and expansion of cells of the ICM. The ICM emerged from the embryo as a large compact colony growing under the zona pellucida and forming an outgrowth with undefined shape and clear borders (FIG. 1). Embryos from the second group did not attach within 5 to 7 days in culture although in vitro maturation and expansion of cell of the ICM were observed. To facilitate hatching, the zona pellucida from these embryos was gently split apart using fine needles and completely removed from the culture. Released cells of the ICM were collected and plated onto fresh feeders. Small, compact ES cell-like colonies with distinct boundaries were detected 3-5 days after replating. A third group of embryos that was not manipulated mechanically was subjected to regular media replacement and allowed to hatch spontaneously. Five of 7 embryos from this group hatched and produced embryo-derived outgrowth but subsequent colonies grew slowly and degenerated or differentiated after two to four passages. In summary, of the 69 embryos collected in this study, 24 hatched and produced large outgrowths which were transferred onto fresh feeders five to ten days after initiation of the cultures.

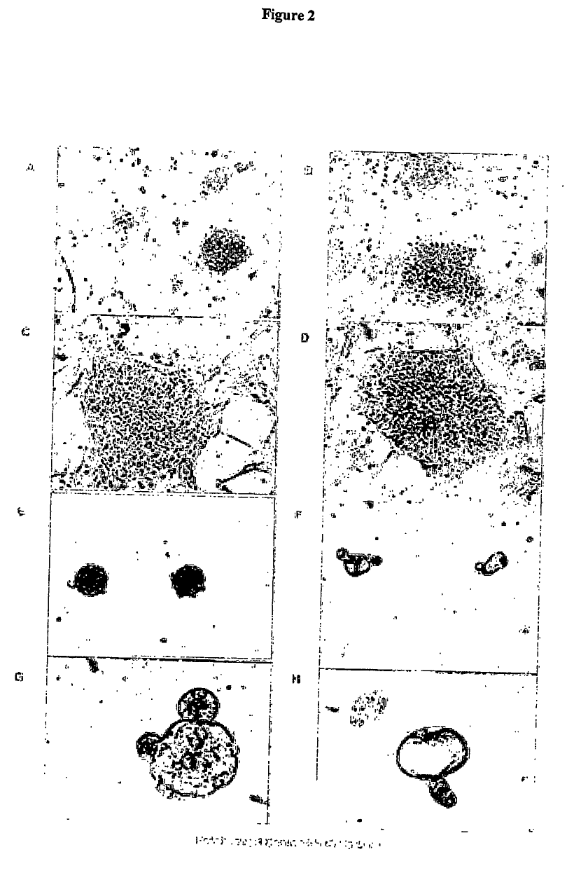

Transfer by mechanical cutting and gentle desegregation of colonies with or without 0.125% dispase or 0.1% collagenase/dispase resulted in the establishment of colonies with ES cell-like morphology after several days. In contrast, exposure of embryo-derived outgrowths to enzymes such as 0.05-0.25% trypsin, 1% dispase or 0.1% collagenase type IV, increased cell death and loss of undifferentiated ES cell colonies. Two to 3 mechanical transfers were required to propagate sufficient number of ES-like colonies. In total, 12 independent ES cell-like lines were generated. However, eight were subsequently lost during in vitro manipulation. The remaining four canine ES cell lines were passaged every 3 to 7 days by exposure to either 0.1% collagenase/dispase or 0.125% dispase. Two phenotypically distinguishable colony types were detected. Some colonies grew as tightly packed bundles of cells with dark nucleoli, distinct borders with a 3-D appearance resembling mouse ES cells colonies. The second colony type were larger with a more flattened appearance more reminiscent of human ES cell colonies. These colonies expanded more rapidly than the tightly packed 3-D colonies (FIG. 2). Canine ES cells with an ES-like morphology have been maintained in vitro for at least 4 months. In general, the morphology of the ES colonies and their ability to proliferate in an undifferentiated state is likely related to the initial developmental stage of the embryo from which the ES cells are derived. Furthermore, expanded blastocysts show the greatest potential to generate ES cell-like colonies. This may be due to an increased number of pluripotent cells in the ICM at that stage of embryo development.

Cryopreservation of ES cells

Canine ES cell lines were successfully cryopreserved and thawed. Removal of DMSO after thawing by washing in complete DMEM/F12 supplemented with either 30% dog serum or FCS was critical for survival of ES cells. Significant cell death and loss of ES-like colonies were noted when cells were thawed and plated in the presence of DMSO. Cells were plated in sufficient volume of medium to cover the bottom of the culture dish and media was replaced after several hours. ES cells surviving cryopreservatio and thawing retained an undifferentiated canine ES phenotype with respect to cell morphology and cellular proliferation.

Expression of Embryonic Stem Cell Markers

Three canine ES cell lines were studied for expression of embryonic stem cell antigens indicative of an undifferentiated state. Antigen expression on canine ES cells was compared with expression on murine ES cells and expression profiles reported for human ES cells. Canine ES cells expressed alkaline phosphatase all-be-it at levels lower than that detected in murine ES cells. AP activity has been demonstrated in pluripotent stem cells of mouse and human origin (46, 47). Similar to human and primate ES cells (48-53), canine ES cells expressed the cell surface marker stage-specific embryonic antigen-4 (SSEA-4) and TRA-1-60 but did not react with the SSEA-1 antibody. In contrast, murine ES cells expressed SSEA-1 but did not react with the SSEA-4 antibody. A third canine ES cell line that had lost the undifferentiated phenotype during in vitro expansion, did not react with the SSEA-4 and TRA-1-60 antibodies.

Expression of murine and human Oct-4, a POU domain transcription factor, is largely restricted to pluripotent stem cells of the inner cell mass(54-60). Oct4 expression is downregulated as these cells differentiate to trophoblast stem cells and derivatives of the three embryonic germ layers. Oct-4 expression was examined by RT-PCR in undifferentiated canine ES cells at first passage, after ten in vitro passages, in differentiated canine ES cells and in mouse ES and TS cells. A 119 bp Oct-4 fragment, which was confirmed by DNA sequencing, was detected in both early and late passage undifferentiated canine ES cells and in mouse ES cells. Oct4 expression was downregulated in murine TS cells and expression was not detected in differentiated canine ES cells. Thus Oct4 expression, which is essential for the maintenance of an embryonic stem cell phenotype, confirms the undifferentiated phenotype of the established canine ES cell lines.

Generation of Embryoid Bodies

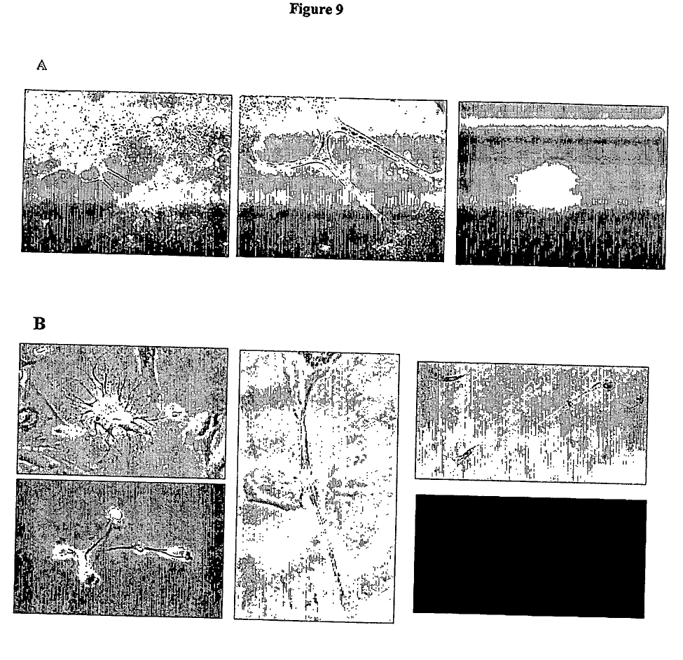

Plating canine ES cells at low density or as single cell suspensions in the absence of feeder layers resulted in their differentiation or death. Transfer of single cell suspensions or small clumps of cells from ES cell colonies to a sparse layer of MEFs or gelatinized dishes resulted in the formation of structures resembling embryoid bodies (EBs). After transfer to non-coated culture dishes, EBs enlarged and developed cystic formations (FIG. 2). Embryoid bodies contain all tissue types and can be further manipulated to generate differentiated cells including skin, muscle, bone, and neurons. The development of EBs from undifferentiated canine ES cells to thus indicate that the canine ES cells have retained the potential to differentiate in vitro to multiple tissue types. EBs were differentiated to neuronal cells and endothelial cells (FIG. 9). Undifferentiated ES cells were also differentiated into cardiomyocytes as evidenced by beating or pulsing (i.e. spontaneously contracting) cultures.

Summary

In 9 separate experiments embryos were collected on days 9-13 post-insemination. An average of 8 viable embryos were obtained per experiment ranging from late morula/early blastocysts (day 9) to early/expanded blastocysts (day 11) or primarily hatched blastocysts (day 13). Embryos were maintained on feeder layers that were either mouse embryonic fibroblasts (MEFs) or canine embryonic fibroblast-like cells (CEFLs) under conditions used for the expansion of mouse or human ES cells.

Both MEFs and CEFLs supported the development of compact morula to early, expanded and hatched blastocysts. Optimal development of morula to the blastocyst stage occurred in medium supplemented with canine serum and under culture conditions similar to those used for the generation of human ES cell lines. Hatching of embryos from zona pellucida occurred either spontaneously or by mechanical cutting. Blastocysts spontaneously hatched 5 to 10 days after initiation of cultures with cells of the inner cell mass expanding and adhering to feeders. Three to 5 days after hatching, inner cell mass outgrowths were mechanically transferred to new feeders at high density with large flat colonies appearing 5-7 days after transfer.

Canine ES cells have been maintained in vitro in an undifferentiated state for five months. Plating of ES cells in the absence of fibroblasts, at low density or as single cells resulted in their differentiation or death. Transfer of single cell suspensions or small clumps of cells from ES colonies to a sparse layer of MEFs resulted in the formation of embryoid bodies (EBs) with cystic formations developing after transfer to noncoated culture dishes.

### CONCLUSIONS

Results showed that canine embryos developed to the compact morula stage by day 10 post-ovulation and by day 12-13 the majority of embryos had hatched. In addition, it was observed that the developmental stage of harvested embryos from the same or different females often varied.

It is important to collect embryos at 10-11 days post ovulation to assure maximum embryo recovery and in vitro maturation.

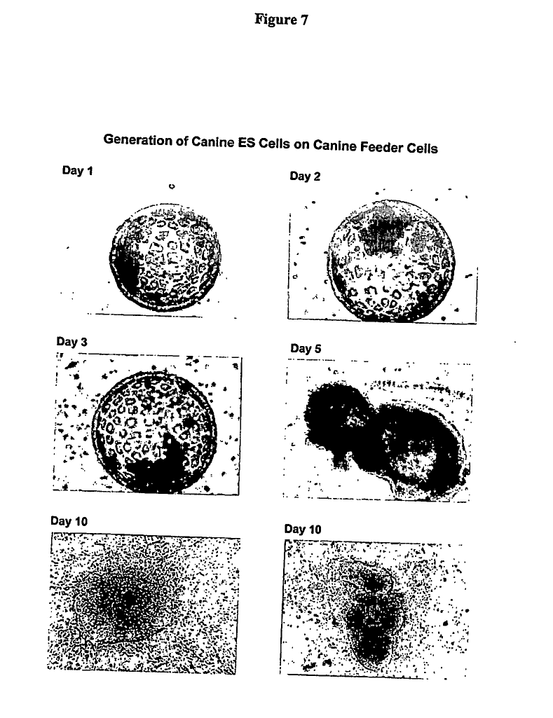

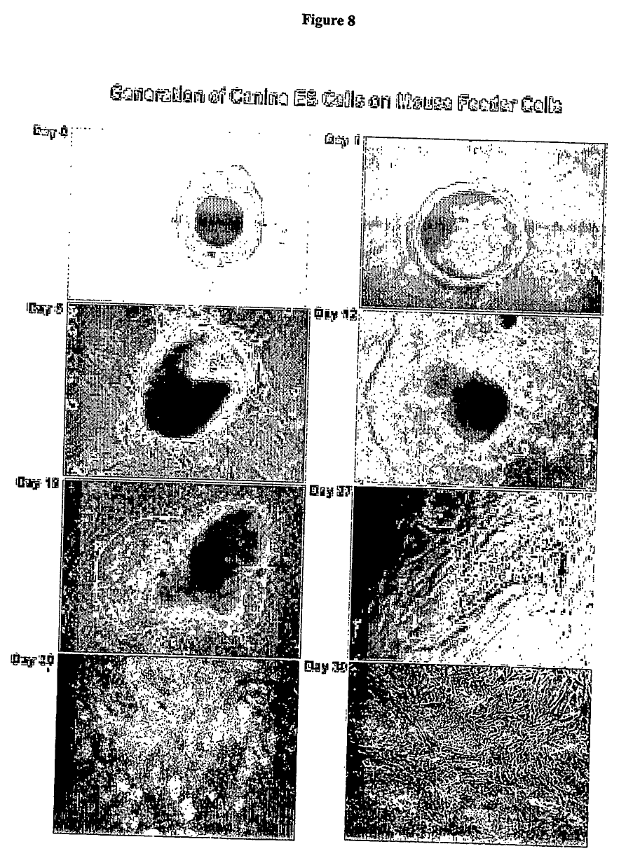

Although, CEF-like cells enabled more rapid expansion and hatching, embryo-derived ICM outgrowths did not result in the appearance of cells with an ES phenotype. (FIG. 7). Optimal development of canine embryos occurred using MEF feeder layers (FIG. 8) and culture conditions similar to those used for the generation of human ES cell lines in media supplemented with either batch tested FBS or canine serum.

Embryo hatching was achieved either spontaneously or by mechanical manipulation. However, only outgrowths from manipulated embryos gave rise to ES-like cells.

If mechanical cutting and gentle disaggregation into small pieces was used as a transfer method, colonies with ES cell-like morphology appeared several days after transfer. Treatment of colonies with 0.125% dispase or 0.1% collagenase/dispase also facilitated the disaggregation of colonies to small clusters of cells, and expansion of ES cell colonies in an undifferentiated state. However, exposure of embryo-derived outgrowths to enzymes such as 0.05% trypsin, 0.25% trypsin, 1% dispase or 0.1% collagenase type IV, increased cell death and inhibited the development and propagation of ES cell-like colonies. Similar to human ES cell lines, canine ES cells must be passaged at a very high density for maintenance of the undifferentiated phenotype.

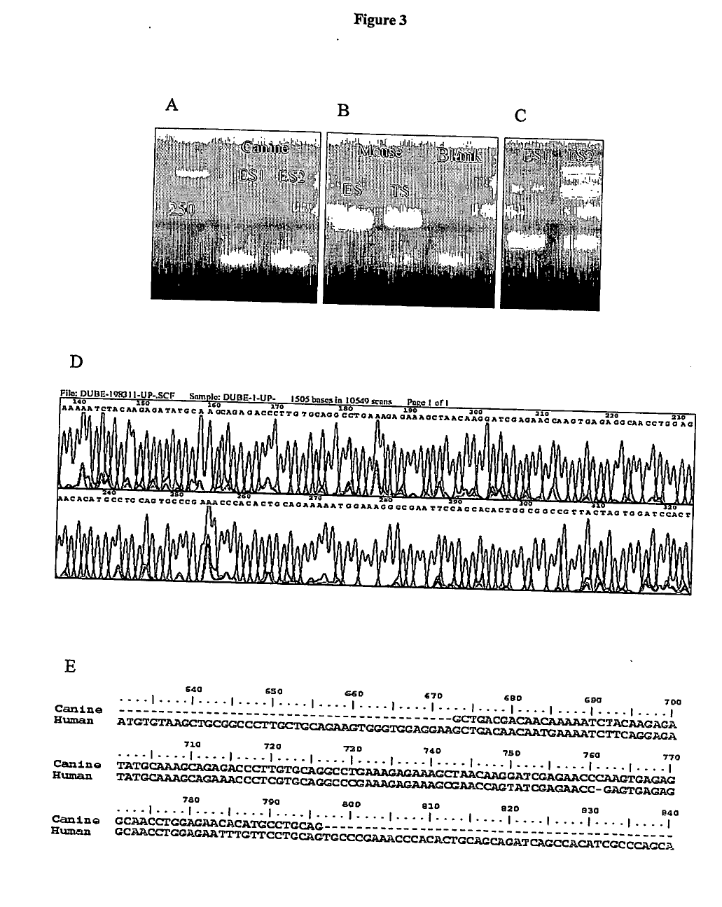

Characterization of canine ES cell colonies indicated that canine ES cells express the Oct-4 transcription factor and cell surface antigens including SSEA-4 and TRA-1-60. Low-level AP expression was also seen and expression of SSEA-1 was not detected (FIGS. 3 -6). These findings indicate that the canine ES cells could be maintained in an undifferentiated state during multiple in vitro passages in culture. Moreover, the phenotype of canine ES cells is more similar to human than murine ES cells

Canine ES cell lines have been cryopreserved and successfully recovered and expanded after cryopreservation.

Transfer of single cell suspensions or small clumps of cells from ES colonies to a sparse layer of MEFs or gelatinized dishes resulted in the formation of structures resembling embryoid bodies (EBs). After transfer to non-coated culture dishes, EBs developed cystic formations. Canine ES cells can also be differentiated in vitro to endothelial and neuronal cells.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

TABLE 1
Identification of Culture Media Supporting Canine Embryo Attachment
and Hatching

| | Number or Embryos from Dog 1 | | |
|---|---|---|---|
| Culture Conditions | TOTAL | ATTACHED | HATCHED |
| DMEM/F12 + FBS | 2 | 2/2 | 2/2 |
| KO DMEM + FBS | 2 | 2/2 | ½ |
| DMEM/F12 + SR | 2 | 0/2 | 0/2 |
| KO DMEM + SR | 2 | 2/2 | 0/2 |

TABLE 2
Embryonic Developmental Stage at the Time of Embryo Harvest

| | Days | | | | |
|---|---|---|---|---|---|
| | Post-ovulation | | | | |
| | 9 | 10 | 11 | 12-13 | Total # |
| | Dog 5 | Dog 4 | Dog 3 | Dog 2 | Of embryos |
| 16-cell Embryo | 4 | | | | 4 |
| Compact Morula | | 9 | | | 8 |
| Early Blastocyst | | | 4 | | 4 |
| Expanded | | | | 2 | 2 |
| Blastocyst | | | | | |
| Hatching Blastocyst | | | | 6 | 6 |
| Total # of embryos | 4 | 9 | 4 | 8 | 25 |

TABLE 3
Embryonic Developmental Stage Optimal for In Vitro Expansion

| | Embryonic | | | | |
|---|---|---|---|---|---|
| Day Post- | Developmental | N^{o} of Embryos | | | |
| Ovulation | Stage | Total | Attached | Expanded | Hatched |
| 9 | 16-cell Embryo | 4 | ¼ | 0/4 | 0/4 |
| 10 | Compact Morula | 9 | 7/9 | 9/9 | 9/9 |
| 11 | Early/Expanded | 4 | 4/4 | ¾ | ¾ |
| | Blastocysts | | | | |
| 12-13 | Expanded Hatching | 8 | ⅜ | 0/8 | 0/8 |
| | Blastocysts | | | | |

TABLE 4
Hatching and Expansion of ICMs

| | Compact | | |
|---|---|---|---|
| | Morula | Early Blastocyst | Expanded Blastocyst |
| Zona Pellucida | 2 | 0 | 5 |
| Cut-Open | | | |
| Zone Pellucida | 0 | 2 | 5 |
| Removed | | | |
| No Manipulation | 1 | 2 | 4 |

### REFERENCES

1. Evans, M. J. & Kaufman, M. H. (1981) Nature 292, 154-6.
2. Andrews, P. W., Damjanov, I., Simon, D., Banting, G. S., Carlin, C., Dracopoli, N. C. & Fogh, J. (1984) Lab Invest 50, 147-62.
3. Bradley, A., Evans, M., Kaufman, M. H. & Robertson, E. (1984) Nature 309, 255-6.
4. Keller, G. M. (1995) Curr Opin Cell Biol 7, 862-9.
5. Maltsev, V. A., Rohwedel, J., Hescheler, J. & Wobus, A. M. (1993) Mech Dev 44, 41-50.
6. Lee, S. H., Lumelsky, N., Studer, L., Auerbach, J. M. & McKay, R. D. (2000) Nat Biotechnol 18, 675-9.
7. Assady, S., Maor, G., Amit, M., Itskovitz-Eldor, J., Skorecki, K. L. & Tzukerman, M. (2001) Diabetes 50, 1691-7.
8. Lumelsky, N., Blondel, O., Laeng, P., Velasco, I., Ravin, R. & McKay, R. (2001) Science 292, 1389-94.
9. Jones, E. A., Tosh, D., Wilson, D. I., Lindsay, S. & Forrester, L. M. (2002) Exp Cell Res 272, 15-22.
10. Harnazaki, T., Iiboshi, Y., Oka, M., Papst, P. J., Meacham, A. M., Zon, L. I. & Terada, N. (2001) FEBS Lett 497, 15-9.
11. Sottile, V., Thomson, A. & McWhir, J. (2003) Cloning Stem Cells 5, 149-55.
12. Zur Nieden, N. I., Kempka, G. & Ahr, H. J. (2003) Differentiation 71, 18-27.
13. Buttery, L. D., Bourne, S., Xynos, J. D., Wood, H., Hughes, F. J., Hughes, S. P., Episkopou, V. & Polak, J. M. (2001) Tissue Eng 7, 89-99.
14. Dani, C. (1999) Cells Tissues Organs 165, 173-80.
15. Dani, C., Smith, A. G., Dessolin, S., Leroy, P., Staccini, L., Villageois, P., Darimont, C. & Ailhaud, G. (1997) J Cell Sci 110, 1279-85.
16. Thomson, J. A., Itskovitz-Eldor, J., Shapiro, S. S., Waknitz, M. A., Swiergiel, J. J., Marshall, V. S. & Jones, J. M. (1998) Science 282, 1145-7.
17. Reubinoff, B. E., Pera, M. F., Fong, C. Y., Trounson, A. & Bongso, A. (2000) Nat Biotechnol 18, 399-404.
18. Kaufman, D. S., Hanson, E. T., Lewis, R. L., Auerbach, R. & Thomson, J. A. (2001) Proc Natl Acad Sci U S A 98, 10716-21.
19. Odorico, J. S., Kaufman, D. S. & Thomson, J. A. (2001) Stem Cells 19, 193-204.
20. Li, F., Lu, S., Vida, L., Thomson, J. A. & Honig, G. R. (2001) Blood 98, 335-42.
21. Zhang, S. C., Wernig, M., Duncan, I. D., Brustle, O. & Thomson, J. A. (2001) Nat Biotechnol 19, 1129-33.
22. He, J. Q., Ma, Y., Lee, Y., Thomson, J. A. & Kamp, T. J. (2003) Circ Res 93, 32-9.
23. Kaufman, D. S. & Thomson, J. A. (2002) J Anat 200, 243-8.
24. Reubinoff, B. E., Itsykson, P., Turetsky, T., Pera, M. F., Reinhartz, B., Itzik, A. & Ben-Hur, T. (2001) Nat Biotechnol 19, 1134-40.
25. Pera, M. F. (2001) Curr Opin Genet Dev 11, 595-9.
26. Verfaillie, C. M., Pera, M. F. & Lansdorp, P. M. (2002) Hematology (Am Soc Hematol Educ Program), 369-91.
27. Thomson, J. A., Kalishman, J., Golos, T. G., Durning, M., Harris, C. P., Becker, R. A. & Hearn, J. P. (1995) Proc Natl Acad Sci U S A 92, 7844-8.
28. Thomson, J. A., Kalishman, J., Golos, T. G., Durning, M., Harris, C. P. & Hearn, J. P. (1996) Biol Reprod 55, 254-9.
29. Suemori, H., Tada, T., Torii, R., Hosoi, Y., Kobayashi, K., Imahie, H., Kondo, Y., Iritani, A. & Nakatsuji, N. (2001) Dev Day 222, 273-9.
30. Ostrander, E. A., Galibert, F. & Patterson, D. F. (2000) Trends Genet 16, 117-24.
31. Kolb, H. J., Guenther, W., Gyurkocza, B., Hoetzl, F., Simoes, B., Falk, C., Schleuning, M. & Ledderose, G. (2003) Transplantation 75, 26S-31S.
32. Georges, G. E. & Storb, R. (2003) Int J Hematol 77, 3-14.
33. Maris, M. & Storb, R. (2002) Cancer Treat Res 110, 149-75.
34. Lutzko, C., Omori, F., Abrams-Ogg, A. C., Shull, R., Li, L., Lau, K., Ruedy, C., Nanji, S., Gartley, C., Dobson, H., Foster, R., Kruth, S. & Dubé, I. D. (1999) Hum Gene Ther 10, 1521-32.
35. Lutzko, C., Kruth, S., Abrams-Ogg, A. C., Lau, K, Li, L., Clark, B. R., Ruedy, C., Nanji, S., Foster, R., Kohn, D., Shull, R. & Dubé, I. D. (1999) Blood 93, 1895-905.
36. Shull, R., Lu, X., Dubé, I., Lutzko, C., Kruth, S., Abrams-Ogg, A., Kiem, H. P., Goehle, S., Schuening, F., Millan, C. & Carter, R. (1996) Blood 88, 377-9.
37. Bienzle, D., Abrams-Ogg, A. C., Kruth, S. A., Ackland-Snow, J., Carter, R. F., Dick, J. E., Jacobs, R. M., Kamel-Reid, S. & Dubé, I. D. (1994) Proc Natl Acad Sci U S A 91, 350-4.
38. Abrams-Ogg, A. C., Kruth, S. A., Carter, R. F., Dick, J. E., Valli, V. E., Kamel-Reid, S. & Dubé, I. D. (1993) Can J Vet Res 57, 79-88.
39. Abrams-Ogg, A. C., Kruth, S. A., Carter, R. F., Valli, V. E., Kamel-Reid, S. & Dubé, I. D. (1993) Am J Vet Res 54, 635-42.
40. Carter, R. F., Abrams-Ogg, A. C., Dick, J. E., Kruth, S. A., Valli, V. E., Kamel-Reid, S. & Dubé, I. D. (1992) Blood 79, 356-64.
41. Meertens, L., Zhao, Y., Rosic-Kablar, S., Li, L., Chan, K., Dobson, H., Gartley, C., Lutzko, C., Hopwood, J., Kohn, D., Kruth, S., Hough, M. R. & Dubé, I. D. (2002) Hum Gene Ther 13, 1809-20.
42. Lutzko, C., Meertens, L., Li, L., Zhao, Y., Abrams-Ogg, A., Woods, J. P., Kruth, S., Hough, M. R. & Dube, I. D. (2002) Exp Hematol 30, 801-8.
43. Moise, N. S. (1999) Cardiovasc Res 44, 37-46.
44. Smith, G. K. (1997) J Am Vet Med Assoc 210, 1451-7.
45. Roush, J. K. (1993) Vet Clin North Am Small Anim Pract 23, 855-68.
46. Benham, F. J., Andrews, P. W., Knowles, B. B., Bronson, D. L. & Harris, H. (1981) Dev Biol 88, 279-87.
47. Andrews, P. W., Meyer, L. J., Bednarz, K. L. & Harris, H. (1984) Hybridoma 3,33-9.
48. Badcock, G., Pigott, C., Goepel, J. & Andrews, P. W. (1999) Cancer Res 59, 4715-9.
49. Andrews, P. W., Gonczol, E., Fenderson, B. A., Holmes, E. H., O'Malley, G., Hakomori, S. & Plotkin, S. (1989) J Exp Med 169, 1347-59.
50. Andrews, P. W., Damjanov, I., Simon, D. & Dignazio, M. (1985) Differentiation 29, 127-35.
51. Andrews, P. W., Banting, G., Damjanov, I., Arnaud, D. & Avner, P. (1 984) Hybridoma 3, 347-61.
52. Henderson, J. K., Draper, J. S., Baillie, H. S., Fishel, S., Thomson, J. A., Moore, H. & Andrews, P. W. (2002) Stem Cells 20, 329-37.
53. Draper, J. S., Pigott, C., Thomson, J. A. & Andrews, P. W. (2002) J Anat 200, 249-58.
54. Buehr, M., Nichols, J., Stenhouse, F., Mountford, P., Greenhalgh, C. J., Kantachuvesiri, S., Brooker, G., Mullins, J. & Smith, A. G. (2003) Biol Reprod 68, 222-9.
55. Fuhrmann, G., Sylvester, I. & Scholer, H. R. (1999) Cell Mol Biol (Noisy-le-grand) 45, 717-24.
56. Hansis, C., Grifo, J. A. & Krey, L. C. (2000) Mol Hum Reprod 6, 999-1004.
57. Kirchhof, N., Carnwath, J. W., Lemme, E., Anastassiadis, K., Scholer, H. & Niemann, H. (2000) Biol Reprod 63, 1698-705.
58. Palmieri, S. L., Peter, W., Hess, H. & Scholer, H. R. (1994) Dev Biol 166, 259-67.
59. Nichols, J., Zevnik, B., Anastassiadis, K., Niwa, H., Klewe-Nebenius, D., Chambers, I., Scholer, H. & Smith, A. (1998) Cell 95, 379-91.
60. Boiani, M., Eckardt, S., Scholer, H. R. & McLaughlin, K. J. (2002) Genes Dev 16, 1209-19.

1. Pluripotent embryonic stem cells isolated from in vitro treatment of canine embryos.

2. A purified cell preparation comprising cells exhibiting a canine embryonic stem cell phenotype.

3. A purified cell preparation comprised or enriched for canine embryonic stem cells that are capable of indefinite proliferation in vitro in an undifferentiated state.

4. A purified cell preparation as claimed in claim 2 wherein cells are immunoreactive with markers for canine embryonic stem cells.

5. A purified cell preparation as claimed in claim 4 wherein the markers are not found in murine embryonic stem cells.

6. A purified cell preparation as claimed in claim 4 wherein the markers are alkaline phosphatase, AP, stage-specific embryonic antigen-4 (SSEA-4), TRA-1-60, and Oct4 transcription factor.

7. A purified cell preparation as claimed in claim 2 induced to differentiate into cells of various lineages.

8. Cells differentiated in vitro from cells of a cell preparation according to claim 2.

9. A cell line comprising canine embryonic stem cells, or cells differentiated or derived therefrom.

10. Embryonic stem cells or a cell preparation according to claim 2 wherein the cells are genetically modified.

11. A method for producing purified canine embryonic stem cells comprising the step of culturing inner cell mass cells from a canine embryo under conditions to promote proliferation of undifferentiated cells.

12. A method as claimed in claim 11 which further comprises isolating a canine embryo, culturing the embryo in the presence of a feeder layer and one or more proliferation agent, removing a blastocyst outgrowth and culturing the outgrowth in the presence of a fresh feeder layer.

13. A method of claim 11 further comprising: (a) obtaining a canine embryo at a morula to blastocyst stage; (b) removing inner cell mass (ICM) cells from the canine embryo; (c) culturing inner cell mass (ICM) cells in the presence of a feeder layer and one or more proliferation agent to promote proliferation of undifferentiated stem cells; and (c) recovering stem cells.

14. A method according to claim 11, further comprising passaging the stem cells to prevent differentiation of the cells and to maintain a cell line in culture.

15. A method for producing cells exhibiting a canine embryonic stem cell phenotype comprising (a) obtaining a canine embryo at a morula to blastocyst stage; (b) culturing inner cell mass (ICM) cells from the canine embryo under conditions which promote proliferation of undifferentiated stem cells; and (c) recovering stem cells.

16. A method of claim 11 comprising inducing differentiation of the embryonic stem cells into cells that exhibit morphological, physiological, functional, and/or immunological features of somatic and germ cells.

17. A cell preparation or cell line derived from cells cultured in accordance with a method of claim 11.

18. Canine transgenic cells, cell lines, or tissues produced using the canine embryonic stem cells of claim 11.

19. A blastocyst to which has been introduced one or more canine embryonic stem cells of a preparation of claim 11.

20. An embryonic cell to which has been introduced by nuclear transfer a nucleus of an embryonic stem cell of a preparation of claim 11.

21. A chimeric non-human animal which is the progeny of a blastocyst according to claim 19.

22. A method which comprises introducing into a blastocyst one or more stem cells made according to a method of claim 11.

23. A method which comprises introducing by nuclear transfer into an embryonic cell a nucleus of a stem cell according to claim 1.

24. A method which comprises introducing to the uterus of a pseudo-pregnant foster mother animal a viable embryo obtained using a technique involving a method of claim 11.

25. Use of embryonic stem cells or cell preparations according to claim 2 in the preparation of a medicament for cell and gene therapies aimed at alleviating disease.

26. A use of claim 25 wherein the embryonic stem cells or cell preparations are transplanted in, or grafted to a subject in need.

27. Use of embryonic stem cells or cell preparations according to claim 2 in the preparation of a medicament for the replacement of body tissues, organs, components or structures which are missing or damaged due to trauma, age, metabolic or toxic injury, disease, or idiopathic loss.

28. A pharmaceutical composition comprising embryonic stem cells or cell preparations according to claim 2 and a pharmaceutically acceptable carrier, excipient, or diluent.

29. A method for testing toxicity of a drug comprising culturing embryonic stem cells, cell preparations, or cell lines as claimed in claim 2 in a suitable medium, introducing a drug to the culture and examining cells to determine if the drug has had an adverse effect on the cells.

30. A method of screening for potential drugs that modulate canine development comprising incubating a test compound and embryonic stem cells as claimed in claim 2 under conditions sufficient to allow the test compound and stem cells to interact, and determining the effect of the compound on a function of the stem cells before and after incubating with the test compound.

31. A method for assaying the activity of a test substance comprising the steps of: (a) exposing embryonic stem cells, cell preparations or cell lines as claimed in claim 2 to a test substance; and (b) detecting the presence or absence of an effect of the test substance on the survival of the cells or on a morphological, functional, or physiological characteristic and/or molecular biological property of the cells, whereby an effect altering cell survival, a morphological, functional, or physiological characteristic and/or a molecular biological property of the cells indicates the activity of the test substance.

32. A method for screening a potential new drug to treat a disorder comprising the steps of: (a) exposing embryonic stem cells, cell preparations or cell lines as claimed in claim 2 to a potential new drug; and (b) detecting the presence or absence of an effect of the potential new drug on the survival of the cells or on a morphological, functional, or physiological characteristic and/or molecular biological property of said cells, whereby an effect altering cell survival, a morphological, functional, or physiological characteristic and/or a molecular biological property of the cells indicates the activity of the potential new drug.

33. A method of treating a condition or disease in a subject comprising administering an effective amount of embryonic stem cells or cell preparations as claimed in claim 2.

34. A method of claim 33 wherein the condition or disease is hemophilia, muscular dystrophy, MPS-1, glycogen storage disease, narcolepsy, thrombasthenia, Von Willebrand Disease, osteogenesis, nephritis, retinal atrophy, severe combined immunodeficiency disease, hematopoietic disorder, autoimmune disorder, cancer, heart disease, motor neuron disease, degenerative bone and joint diseases, and atherosclerosis.

35. A method for conducting a stem cell business comprising (a) identifying one or more agents which affect the proliferation, differentiation, function, or survival of embryonic stem cells, cell preparations, or cell lines according to claim 2; (b) conducting therapeutic profiling of agents identified in (a); or analogs thereof for efficacy and toxicity in animals; and (c) formulating a pharmaceutical composition including one or more agents identified in (b) as having an acceptable therapeutic profile.

36. A kit comprising embryonic stem cells as claimed in claim 2 and instructions for their use.

37. A primer that hybridizes to an Oct4 canine nucleotide sequence.

38. A primer comprising the sequence of SEQ ID No. 1,2, 5 or 6.
